(19) United States
(12) Patent Application Publication
(10) Pub. No.: US 2019/0183387 A1
(43) Pub. Date: Jun. 20, 2019

(54) SUBJECT DATA MANAGEMENT SYSTEM

(71) Applicant: Impedimed Limited, Pinkenba, Queensland (AU)

(72) Inventors: Jack Gerald Cosentino, Savage, MN (US); Richard Roland Carreon, Fallbrook, CA (US)

(21) Appl. No.: 16/328,088

(22) PCT Filed: Aug. 25, 2017
(86) PCT No.: PCT/AU2017/050901

§ 371 Date: Feb. 25, 2019

Related U.S. Application Data
(60) Provisional application No. 62/380,214, filed on Aug. 26, 2016.

(51) Int. Cl. A61B 5/117; A61B 5/053; A61B 5/00; G16H 10/60; G16H 40/67; G06F 21/60; G06F 16/23

(52) U.S. Cl. CPC A61B 5/117; A61B 5/0537; A61B 5/7264; A61B 5/7435; A61B 5/02444; G16H 40/67; G06F 21/602; G06F 16/2379; G16H 10/60

(57) ABSTRACT

A system for managing subject data relating to a body status of a subject, the system including one or more subject databases storing subject data for a plurality of subjects, the subject data being indicative of body status indicator and measured body parameter values of the subject and one or more processing devices that receives collected subject data from a client device via a communications network, the client device receiving measurement data indicative of at least one measured body parameter value of a subject from a measuring device, and the collected subject data being indicative of at least one of the measurement data and the at least one measured body parameter value and determines an identity of the subject, updates the subject data for the subject using the collected subject data and retrieves retrieved subject data for the subject, the retrieved subject data being indicative of measured body parameter values for the respective subject and being used to derive a body status indicator indicative of a body status of the subject.

### BACKGROUND OF THE INVENTION

The present invention relates to a system and method for managing subject data relating to a body status of a subject and for monitoring a body status of a subject.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Whilst it is known to provide electronic medical records, the uptake and use of such records has been limited for a number of reasons. For example, the sensitivity of information contained in medical records means it is necessary for medical records to be maintained securely, which can be problematic and in particular places burdens on both the holder and users accessing the medical records. Additionally, information is still largely entered into the medical records systems manually. This introduces a bottle neck into the effective creation of medical records, as well as resulting in potential for information to be entered inaccurately, which could in turn have a significant impact on patient outcomes.

### SUMMARY OF THE PRESENT INVENTION

In one broad form an aspect of the present invention seeks to provide a system for managing subject data relating to a body status of a plurality of subjects, the system including: a measuring device that acquires measurement data at least partially indicative of impedance measurements performed on a subject; a client device in communication with the at least one measurement device that: receives measurement data; and determines identity information indicative of an identity of the subject; generates collected subject data indicative of at least one of: the measurement data; and, measured body parameter values of the subject, the measured body parameters being at least partially derived from impedance measurements performed on the subject; and, at least one body status indicator at least partially derived from the measured body parameter values; and, one or more processing devices including: one or more first processing devices that: receive the collected subject data and an indication of the identity information; retrieve an identifier from an index at least in part using the identity information, the index being indicative of a respective identifier associated with each of a plurality of subjects; and, store the collected subject data in one or more first subject databases to allow stored subject data to be used in calculating one or more body status indicators indicative of a body status of the subject; and, one or more second processing devices that: receive at least some subject data and the respective identifier of at least one subject from the one or more first processing devices; and, store the subject data in one or more second subject databases in accordance with the respective identifier, to thereby allowing subject data from a plurality of subjects to be analysed.

In one embodiment the one or more processing devices: determine a body status indicator to be displayed; retrieve at least some of the stored subject data for the subject in accordance with the determined body status indicator; and, at least one of: generate the body status indicator and provides the body status indicator to the client device; and, provide retrieved subject data to the client device, the client device being responsive to the retrieved subject data to generate the body status indicator.

In one embodiment the identity information includes authentication information supplied by the subject and wherein the one or more first processing devices authenticate the subject to determine the identity of the subject.

In one embodiment the client device authenticates the subject using authentication information supplied by the subject and provides the identity information in the form of an indication of the identity of the subject in response to authentication of the subject.

In one embodiment the authentication information includes biometric data received via a biometric data reader on the client device.

In one embodiment: the client device transfers identity information to the one or more first processing devices; receives the respective identifier from the one or more first processing devices; and, transfers the collected subject data together with the respective identifier to the one or more first processing devices.

In one embodiment the client device executes a client device software application that enables encrypted communication with a server application executed by the one or more first processing devices.

In one embodiment the one or more processing devices analyse the subject data by performing machine learning using at least subject data for each of a plurality of subjects in order to derive models that can be used in determining body status indicators from measurement data.

In one embodiment the system further includes one or more third processing devices, and wherein the third processing devices: retrieve at least some of the stored subject data and respective subject identifiers from the one or more second processing devices for each of a number of subjects; use the respective subject identifiers to retrieve healthcare data for each of a number of subjects from the one or more first processing devices; and, analyse the retrieved subject data and healthcare data to derive one or more models for use in determining body status indicators from measurement data.

In one embodiment the one or more first processing devices retrieve healthcare data at least one of: by interfacing with an electronic healthcare record system to retrieve healthcare data relating to a respective subject; and, from the one or more first subject databases.

In one embodiment the subject data further includes: an indication of at least one physical characteristic; an indication of at least one symptom; and, the at least one body status indicator.

In one embodiment the client device presents a user interface to: collect at least one of: authentication information; an indication of at least one physical characteristic; and, an indication of at least one symptom; and, display at least one of: the at least one body status indicator; and, at least one measured body parameter values.

In one embodiment the one or more processing devices generate unique identifiers for each of the plurality of subjects.

In one embodiment the one or more first processing devices periodically upload subject data to the one or more second processing devices for storage in the one or more second databases.

In one embodiment the stored subject data is encrypted within the one or more subject databases and wherein the one or more processing devices includes an encryption module that: encrypts subject data stored in the one or more subject databases; and, decrypts retrieved subject data extracted from the one or more subject databases.

In one broad form an aspect of the present invention seeks to provide a method for managing subject data relating to a body status of a plurality of subjects, the method including: in a measuring device, acquiring measurement data at least partially indicative of impedance measurements performed on a subject; in a client device in communication with the at least one measurement device: receiving measurement data; and determining identity information indicative of an identity of the subject; generating collected subject data indicative of at least one of: the measurement data; and, measured body parameter values of the subject, the measured body parameters being at least partially derived from impedance measurements performed on the subject; and, at least one body status indicator at least partially derived from the measured body parameter values; and,

In one or more processing devices including: one or more first processing devices, the one or more first processing devices: receiving the collected subject data and an indication of the identity information; retrieving an identifier from an index at least in part using the identity information, the index being indicative of a respective identifier associated with each of a plurality of subjects; and, storing the collected subject data in one or more first subject databases to allow stored subject data to be used in calculating one or more body status indicators indicative of a body status of the subject; and, one or more second processing devices, the one or more second processing devices: receiving at least some subject data and the respective identifier for at least one subject from the one or more first processing devices; and, storing the subject data in one or more second subject databases in accordance with the respective identifier, to thereby allowing subject data from a plurality of subjects to be analysed.

In one broad form an aspect of the present invention seeks to provide a system for managing subject data relating to a body status of a plurality of subjects, the system including one or more processing devices including: one or more first processing devices that: receive collected subject data and an indication of identity information from a client device via a communications network, the client device receiving measurement data indicative of at least one measured body parameter value of a subject from a measuring device, the measuring device being adapted for performing impedance measurements and the measurement data being at least partially indicative of impedance measurements performed on the subject, and the collected subject data being indicative of at least one of: the measurement data; and, at least one measured body parameter value derived from the measurement data; and, retrieve an identifier from an index at least in part using the identity information, the index being indicative of a respective identifier associated with each of a plurality of subjects; and, store the collected subject data in one or more first subject databases to allow stored subject data to be used in calculating one or more body status indicators indicative of a body status of the subject; and, upload at least some subject data and a respective identifier for at least one subject to one or more second processing devices for storage in one or more second subject databases in accordance with the respective identifier.

In one broad form an aspect of the present invention seeks to provide a method for managing subject data relating to a body status of a plurality of subjects, method including in one or more processing devices that: receiving collected subject data and an indication of identity information from a client device via a communications network, the client device receiving measurement data indicative of at least one measured body parameter value of a subject from a measuring device, the measuring device being adapted for performing impedance measurements and the measurement data being at least partially indicative of impedance measurements performed on the subject, and the collected subject data being indicative of at least one of: the measurement data; and, at least one measured body parameter value derived from the measurement data; and, retrieving an identifier from an index at least in part using the identity information, the index being indicative of a respective identifier associated with each of a plurality of subjects; and, storing the collected subject data in one or more first subject databases to allow stored subject data to be used in calculating one or more body status indicators indicative of a body status of the subject; and, uploading at least some subject data and a respective identifier for at least one subject to one or more second processing devices for storage in one or more second subject databases in accordance with the respective identifier.

In one broad form an aspect of the present invention seeks to provide a system for managing subject data relating to a body status of a subject, the system including: one or more subject databases storing subject data for a plurality of subjects, the subject data being indicative of at least one of: body status information; and, measured body parameter values of the subject; and, one or more processing devices that: receive collected subject data from a client device via a communications network, the client device receiving measurement data indicative of at least one measured body parameter value of a subject from a measuring device, the measuring device being adapted for performing impedance measurements and the measurement data being at least partially indicative of impedance measurements performed on the subject, and the collected subject data being indicative of at least one of: the measurement data; and, the at least one measured body parameter value; and, determine an identity of the subject; update stored subject data for the subject using the collected subject data and the identity of the subject; and, retrieve at least some stored subject data for the subject, the retrieved subject data being indicative of previously measured body parameter values for the respective subject and being used to derive at least one body status indicator indicative of a body status of the subject.

In one embodiment the one or more processing devices: identify a body status indicator to be displayed by the client device; and, determine the retrieved subject data in accordance with the determined body status indicator.

In one embodiment the one or more processing devices at least one of: generate the body status indicator and provide the body status indicator to the client device; and, provide retrieved subject data to the client device, the client device being responsive to the retrieved subject data to generate the body status indicator.

In one embodiment the one or more processing devices at least one of retrieve and store subject data in response to authentication of the subject.

In one embodiment the one or more processing devices: receive authentication information from the client device; compare the authentication data to reference authentication data stored in a memory; and, selectively authenticate the subject depending on a result of the comparison.

In one embodiment the authentication information includes biometric data received via a biometric data reader on the client device.

In one embodiment the one or more processing devices store an index identifying authorised users associated with the subject, to thereby allow access to retrieved subject data by authorised users.

In one embodiment the authorised users correspond to healthcare users.

In one embodiment the authorised users have respective access permissions and wherein the retrieved subject data is determined in accordance with the access permissions of the authorised user.

In one embodiment the one or more processing devices: receive a subject data request from a user client device; and, selectively provide retrieved subject data to the user client device in response to the subject data request.

In one embodiment the one or more processing devices: determine an identity of a user from the subject data request; determine if the user is a selected authorised user; and, selectively provide retrieved subject data to the user client device in response to a positive determination.

In one embodiment the one or more processing devices: receive a subject data request from a user client device relating to a group of one or more subjects; generate a query to query subject data stored in one or more subject databases; retrieve subject data from the one or more subject databases using the query; apply a de-identification algorithm to the retrieved subject data; and, provide de-identified data to the user client device.

In one embodiment the subject data is encrypted within the one or more subject databases and wherein the one or more processing devices includes an encryption module that: encrypts subject data stored in the one or more subject databases; and, decrypts retrieved subject data extracted from the one or more subject databases.

In one embodiment the system includes: one or more first processing devices that: determine identity information indicative of an identity of the subject; retrieve an identifier from an index at least in part using the identity information, the index being indicative of a respective identifier associated with each of a plurality of subjects; and, store the collected subject data in one or more first subject databases to allow stored subject data to be used in calculating one or more body status indicators indicative of a body status of the subject; and, one or more second processing devices that: receive at least some subject data and a respective identifier for at least one subject from the one or more first processing devices; and, store the subject data in one or more second subject databases in accordance with the respective identifier, to thereby allowing subject data from a plurality of subjects to be analysed.

In one embodiment the client device provides the identity information to the one or more first processing devices, the identity information including authentication information supplied by the subject and wherein the one or more first processing devices authenticate the subject using the authentication information to determine the identity of the subject.

In one embodiment the client device authenticates the subject using authentication information supplied by the subject and provides identity information in the form of an indication of the identity of the subject to the one or more first processing devices in response to authentication of the subject.

In one embodiment: the client device transfers identity information to the one or more first processing devices; receives the respective identifier from the one or more first processing devices; and, transfers the subject data together with the respective identifier to the one or more first processing devices.

In one embodiment the system further includes one or more third processing devices, and wherein the third processing devices: retrieve subject data and respective subject identifiers from the one or more second processing devices for each of a number of subjects; use the respective subject identifiers to retrieve healthcare data for each of a number of subjects from the one or more first processing devices; and, analyse the subject data and healthcare data to derive one or more models for use in determining body status indicators from measurement data.

In one embodiment the one or more first processing devices retrieve healthcare data at least one of: by interfacing with an electronic healthcare record system to retrieve healthcare data relating to a respective subject; and, from the one or more first subject databases.

In one embodiment the one or more processing devices generate unique identifiers for each of the plurality of subjects.

In one embodiment the one or more first processing devices periodically uploads the subject data to the one or more second processing devices for storage in the one or more second databases.

In one embodiment the client device presents a user interface to: collect at least one of: authentication information; an indication of at least one physical characteristic; and, an indication of at least one symptom; and, display at least one of: the at least one body status indicator; and, at least one measured body parameter values.

In one embodiment the client device executes a client device software application that enables encrypted communication with a server application executed by the one or more first processing devices.

In one embodiment the one or more processing devices analyse the subject data by performing machine learning using at least subject data for each of a plurality of subjects in order to derive models that can be used in determining body status indicators from measurement data.

In one broad form an aspect of the present invention seeks to provide a system for monitoring a body status of a subject, the system including a client device having: an input; a display; a memory including a software application; and, a client device processor that: receives measurement data indicative of at least one measured body parameter value of the subject from a measuring device; determines identity information indicative of an identity of the subject; provides collected subject data to one or more remote processing devices, the collected subject data being indicative of at least one of: the measurement data; and, at least one measured body parameter value derived from the measurement data, the one or more remote processing devices being responsive to the collected subject data to update subject data stored in one or more subject databases, the subject data indicative of: body status indicator; and, measured body parameter values of the subject; and, displays an indication of a body status indicator derived at least in part from measured body parameter values.

In one embodiment the client device acquires authentication information from the subject via the input, the authentication information being used to authenticate the subject.

In one embodiment the client device authenticates the subject by: comparing the authentication data to reference authentication data stored in the memory; and, selectively authenticating the subject depending on a result of the comparison.

In one embodiment the client device processor: receives retrieved subject data from the one or more processing devices; and, uses the retrieved subject data to generate to the body status indicator.

In one embodiment the client device receives the subject data by providing a subject data request to the one or more processing devices, the one or more processing devices being responsive to the subject data request to provide the subject data to the client device.

In one embodiment the client device receives the body status indicator from the one or more processing devices.

In one embodiment the client device processor receives an indication of at least one measured body parameter value from the measuring device.

In one embodiment the client device: displays a question to the subject; and, determines an indication of at least one response in accordance with subject input commands

In one embodiment the client device processor: determines an indication of one or more selected authorised users in accordance with subject input commands; and, causes the remote processing device to record an indication of the one or more selected authorised users associated with the respective subject.

In one embodiment the client device processor: retrieves an indication one or more authorised users from the remote processing device; displays a list of the one or more authorised users; and, determines selection of the one or more selected authorised users in accordance with subject input commands.

In one embodiment the client device includes: a first wireless communications module that allows the client device to communicate with the measurement device; and, a second wireless communications module that allows the client device to communicate with the remote processing device via the communications network.

In one embodiment the client device is configured to communicate with one or more other client devices using the communications module.

In one embodiment the client device includes at least one of: a smart phone; and, a tablet.

In one embodiment the client device includes a camera to allow video communications.

In one embodiment the client device communicates with the measuring device to cause a measurement procedure to be performed.

In one embodiment the client device causes a measurement procedure to be performed in response to successful authentication of the subject.

In one embodiment the client device: determines a measurement process to be performed; and, instructs the measuring device to perform the selected measurement process.

In one embodiment the system determines an identity of the subject at least in part through authentication of the subject.

In one embodiment the system at least partially analyses the measurement data to determine at least one of: the at least one measured body parameter value; and, a body status indicator.

In one embodiment the system determines the body status indicator by comparing the at least one measured body parameter value to at least one of: a reference range; and, a previously measured body parameter value.

In one embodiment the system: compares at least one of a body status indicator and at least one measured body parameter value to notification criteria; and, selectively generates a notification in response to results of the comparison.

In one embodiment the notification criteria includes: at least one of a body status indicator and at least one measured body parameter value falling outside a reference range; and, a change in at least one of a body status indicator and at least one measured body parameter value falling outside a reference range.

In one embodiment the notification is provided to at least one of: the subject; and, a selected authorised user.

In one embodiment the collected subject data further includes: an indication of at least one physical characteristic; an indication of at least one symptom; and, the at least one body status indicator.

In one embodiment the healthcare data is at least in part determined from a healthcare record.

In one embodiment communication between the one or more processing devices and client device is encrypted.

In one embodiment encryption is performed using at least one of: a session key; a private public key pair associated with the one or more processing devices; and, a private public key pair associated with the client device.

In one embodiment the measuring device includes: a plurality of electrodes provided in electrical contact with the subject in use; at least one sensor coupled to a number of the plurality of electrodes, the at least one sensor being adapted to measure at least one body signal in the subject via the number of the plurality of electrodes; and, a measuring device processor that: receives an indication of the at least one body signal from the at least one sensor; and, generates measurement data indicative of at least one body parameter value using the at least one body signal.

In one embodiment the measuring device includes: a plurality of electrodes provided in electrical contact with the subject in use; at least one signal generator coupled to first subset of the plurality of electrodes, the at least one signal generator being adapted to generate a drive signal which is applied to the subject via the first subset of the plurality of electrodes; at least one sensor coupled to a second subset of the plurality of electrodes, the at least one sensor being adapted to measure at least one response signal in the subject via the second subset of the plurality of electrodes; and, a measuring device processor that: controls the at least one signal generator; receives an indication of a measured at least one response signal from the at least one sensor; and, generates measurement data indicative of at least one body parameter using the at least one response signal, the at least one body parameter including an impedance value.

In one embodiment the measuring device includes a wireless communications module that allows the measuring device to communicate with the client device.

In one embodiment the measuring device processor at least partially processes the at least one body signal to generate the measurement data.

In one embodiment the measurement data includes an indication of voltage signals measured via electrodes in contact with the subject and wherein the at least one body parameter includes at least one of: a respiration parameter; a cardiac parameter; and, an impedance parameter.

In one broad form an aspect of the present invention seeks to provide a system for managing subject data relating to a body status of a subject, the system including: one or more subject databases storing subject data for a plurality of subjects, the subject data being indicative of: body status indicator; and, measured body parameter values of the subject; and, one or more processing devices; and, a client device having: an input; a display; a memory including a software application; and, a client device processor that: receives measurement data indicative of at least one measured body parameter value of the subject from a measuring device; determines an identity of the subject; provides collected subject data to the one or more processing devices, the collected subject data being indicative of at least one of: the measurement data; and, the at least one measured body parameter value, the remote processing device being responsive to the collected subject data to: update subject data stored in the one or more subject databases; in response to a request, retrieve retrieved subject data for the subject, the retrieved subject data being indicative of measured body parameter values for the respective subject and being used to derive a body status indicator indicative of a body status of the subject; and, displays an indication of a body status indicator derived at least in part from measured body parameter values.

In one broad form an aspect of the present invention seeks to provide a method for managing subject data relating to a body status of a subject, the method including, in an electronic processing device: receiving collected subject data from a client device via a communications network, the client device receiving measurement data indicative of at least one measured body parameter value of a subject from a measuring device, and the collected subject data being indicative of at least one of: the measurement data; and, the at least one measured body parameter value; and, determining an identity of the subject; updating subject data for the subject using the collected subject data, the subject data being stored in one or more subject databases and being indicative of: body status indicator; and, measured body parameter values of the subject; and, in response to a request, retrieving retrieved subject data for the subject, the retrieved subject data being indicative of measured body parameter values for the respective subject and being used to derive a body status indicator indicative of a body status of the subject.

In one broad form an aspect of the present invention seeks to provide a method for monitoring a body status of a subject, the method including, in a client device processor of a client device: receiving measurement data indicative of at least one measured body parameter value of the subject from a measuring device; determining an identity of the subject; providing collected subject data to the remote processing device, the collected subject data being indicative of at least one of: the measurement data; and, the at least one measured body parameter value, the remote processing device being responsive to the collected subject data to update subject data stored in one or more subject databases, the subject data indicative of: body status indicator; and, measured body parameter values of the subject; and, displaying an indication of a body status indicator derived at least in part from measured body parameter values.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

An example of the present invention will now be described with reference to the accompanying drawings, in which:

FIG. 1 is a flow chart of an example of a process for managing subject data and monitoring a body status of a subject;

FIG. 2 is a schematic diagram of an example of a system for managing subject data and monitoring a body status of a subject;

FIG. 3 is a schematic diagram of an example of a measuring system;

FIG. 4 is a schematic diagram of an example of a processing system;

FIG. 5 is a schematic diagram of an example of a client device;

FIGS. 6A and 6B are a flow chart of an example of a process for updating subject data;

FIG. 7A is a flow chart of a first example of a process for displaying a body status indicator;

FIG. 7B is a flow chart of a second example of a process for displaying a body status indicator;

FIG. 8 is a flow chart of an example of a process for nominating authorised users;

FIG. 9 is a flow chart of an example of a process for accessing subject data;

FIG. 10 is a flow chart of an example of a process for querying subject data;

FIG. 11 is a flow chart of an example of a process for encrypting communications;

FIG. 12 is a schematic diagram of a specific example hardware architecture;

FIG. 13 is a flow chart of an example of the operation of the hardware of FIG. 12; and,

FIGS. 14A to 14C is a flow chart of a specific example of the operation of the hardware of FIG. 12.

### DETAILED DESCRIPTION OF THE PREFERRED EMBODIMENTS

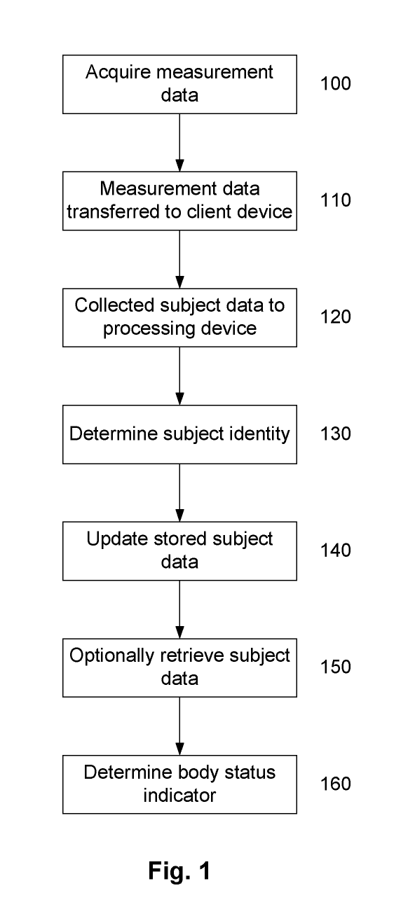

An example of a process for managing subject data relating to a body status of a subject will now be described with reference to FIG. 1.

For the purpose of this example it is assumed that the process is performed at least in parts using one or more electronic processing devices, typically forming part of one or more processing systems, such as servers or the like. The one or more processing devices may be connected to one or more client devices, such as mobile phones, tablets, portable computers, or the like, which are in turn connected to measuring devices, as will be described in more detail below. For the purpose of the following, the term processing device will generally be used to encompass one or more processing devices for the ease of explanation.

In this example, at step 100 measurement data indicative of at least one measured body parameter value is acquired by the measuring device. The measurement device and associated measurement data can be of any appropriate form depending on the preferred implementation. In one example, the measurement device is adapted to measure electrical signals within a subject's body, and can be used to measure body parameter values, such as impedance values, cardiac parameters, respiratory parameters, or the like. The measured electrical signals are used to generate the measurement data, which can therefore include an indication of the raw measured signals and/or parameter values derived therefrom.

At step 110, measurement data is transferred to a client device, such as a mobile phone, tablet, personal computing device, or the like, from the measuring device. This may be achieved in any appropriate manner that typically involves wirelessly transmitting measurement data from the measuring device to the client device, as will be described in more detail below.

At step 120, collected subject data is provided to the one or more processing devices from the client device, via a communications network. This may be achieved in any appropriate manner but typically involves having the client device upload the measurement data to the one or more processing devices, with this generally being performed via a secure connection. The collected subject data can be of any appropriate form, but is typically indicative of either the measurement data and/or one or more measured body parameter values derived therefrom. The collected subject data may also include additional information, such as details of any symptoms suffered by the subject, details of physical characteristics of the user, or the like, as will be described in more detail below.

At step 130 the one or more processing devices determines an identity of the subject. This can be achieved in any appropriate manner and may be based on the collected subject data, or alternatively on other information provided by the client device. In one particular example, this is performed based on authentication of the subject by either the client device or the one or more processing devices. Identifying of the subject is performed in order to allow the one or more processing devices to update stored subject data for the respective subject at step 140. In this regard, the subject data is stored in one or more subject databases that stores subject data for a plurality of subjects. The subject data is of any suitable and generally includes one or more measured body parameter values for the subject, as well as body status indicator, characteristic data indicative of subject characteristics, or the like. Thus, the one or more processing devices updates the subject data by adding the collected subject data to the stored subject data, so the stored subject data includes the measured body parameter values and/or measurement data.

At step 150, the one or more processing devices can optionally retrieve subject data, for example in response to a request from the client device that provided the collected subject data, or from another client device. The retrieved subject data typically represents a subset of the stored subject data, and may be indicative of previous measured body parameter values for the respective subject. The retrieved subject data can be used to derive a body status indicator indicative of a body status of the subject at step 160, for example to allow changes in body parameter values over time to be monitored.

In this regard, the body status indicator could be of any appropriate form and could include a wellness or health status indicator, or a specific indicator such as a heart rate, relative fluid levels, body fat levels, body composition parameters, or the like. The body status indicator, could be based solely on the basis of a current measurement but more typically takes into account previous measurements, thereby looking at changes in heart rate, fluid levels or the like. In one particular example, the body status indicator can be determined by comparing the current or changes in measured body parameter values to a reference range, although other suitable approaches could be used.

Whilst steps 150 and 160 are described as following steps 120 to 140, it will be appreciated that this is not essential and alternatively, steps 150 and 160 can be performed prior to or concurrently with steps 120 to 140, for example allowing previous measured body parameter values to be retrieved and used to calculate a body status indicator, prior to providing the collected subject matter data. This can be used to allow the body status indicator to be displayed to the subject, whilst the collected subject data is being prepared and provided to the one or more processing devices.

In any event, it will be appreciated that the above-described system utilises measurement data obtained directly from a measurement device, with this being used by a client device to generate collected subject data, which is then used to update stored subject data stored centrally in one or more subject databases. The subject data contains information regarding the body status of the subject, including the measured parameter values collected over time and can therefore act as a medical healthcare record, with measured parameter values being stored as part of the healthcare record directly from a measurement device. This obviates problems associated with manual data entry, whilst assisting to ensure data integrity and security, by allowing for secure communication between the one or more processing devices and the client device.

A number of further features will now be described.

In one aspect, the system for managing subject data relating to a body status of a subject includes one or more subject databases storing subject data for a plurality of subjects, the subject data being indicative of body status indicator and measured body parameter values of the subject. The system also includes the one or more processing devices that receives the collected subject data from the client device, determines an identity of the subject, updates the subject data for the subject using the collected subject data and in response to a request, retrieves retrieved subject data for the subject, the retrieved subject data being indicative of measured body parameter values for the respective subject and being used to derive a body status indicator indicative of a body status of the subject.

In another aspect, the system includes a client device having an input, a display, a memory including a software application and a client device processor. The client device processor receives the measurement data indicative from the measuring device, determines an identity of the subject, provides collected subject data to the remote processing device, which updates subject data stored in one or more subject databases, and then displays an indication of a body status indicator derived at least in part from measured body parameter values.

In one example, the body status indicators can include, but are not limited to any one or more of:
Body Composition
Dry Lean Mass
Lean Body Mass
Skeletal Muscle Mass
Segmental Lean Analysis
Body Fat Mass
Segmental Fat Analysis
BMI (Body Mass Index)
PBF (Percent Body Fat)
Visceral Fat Area
Visceral Fat Level
Total Body Water
Intracellular Water
Extracellular Water
ECW/TBW
Segmental Body Water
Segmental ECW/TBW
Segmental ICW Analysis
Segmental ECW Analysis
Body-Fat-LBM Control
BMR (Basal Metabolic Rate)
Leg Lean Mass
TBW/LBM
Whole Body Phase Angle
Segmental Phase Angle
Reactance
Impedance of Each Segment per frequency
Body Water Composition History

In one example, the one or more processing devices provides retrieved subject data to the client device, with the client device being responsive to the retrieved subject data to generate the body status indicator. Thus, the client device processor operates to receive retrieved subject data from the one or more processing devices and then uses the retrieved subject data to generate the body status indicator. Alternatively however, the body status indicator could be generated by the one or more processing devices and then provided to the client device allowing the client device to display the body status indicator. It will be appreciated that this obviates the need to provide subject data to the client device, whilst still allowing the body status indicator to be displayed.

In one example, the one or more processing devices identifies a body status indicator that is to be displayed by the client device and then determines the subject data to be retrieved in accordance with the body status indicator. This allows the one or more processing devices to determine what subject data is required in order to allow the respective body status indicator to be determined, and then only retrieve that particular subject data from the stored subject data, thereby limiting the need to retrieve subject data.

The one or more processing devices uses the identity of the subject in order to ensure the collected subject data is added to the correct subject data record in the one or more subject databases, as well as to ensure that the correct subject data is retrieved when body status indicators are to be generated. In one particular example, the one or more processing devices only stores collected subject data or provides retrieved subject data, in response to authentication of the subject, with the authentication process being used to identify the subject and hence ensure subject data cannot be retrieved by an unauthorised user.

Authentication can be performed locally by the client device, or can be performed by having the one or more processing devices receive authentication information from the client device and then authenticate the subject using the authentication information. The use of central authentication by the one or more processing devices is particularly advantageous as this allows a subject to utilise any client device and not one that is specifically configured for the particular subject.

In either case, the client device typically determines authentication information from the subject via an input with the authentication information being used to authenticate the subject, by comparing the authentication information to reference authentication information stored either on the client device or centrally, for example as part of an authentication database. The authentication information can include biometric data received from a biometric reader on the client device, such as a thumb or fingerprint scan, iris scan, facial representation or the like. However, this is not essential and alternatively other forms of authentication information, such as a username and password, personal identification number (PIN), or the like can be used.

In any event, this process allows the subject to undergo authentication via the client device, with retrieved subject data being provided in response to successful authentication. This ensures that the subject is able to successfully retrieve the subject data and prevents the subject's data being used by unauthorised users.

Nevertheless, it can be desirable to allow third parties to access the data, for example allowing subject data to be viewed by healthcare professionals or the like. To achieve this, the one or more processing devices can access an index identifying authorised users associated with the subject, thereby allowing the authorised users access to retrieved subject data. The authorised users typically correspond to healthcare users and in one example have respective access permissions with a subset of the subject data being provided depending on those access permissions. For example, a doctor that is the personal physician of a subject may have access to more of the subject data than a nurse that is assigned to only carry out specific tasks, such as examining blood pressure or the like.

In order to create the index, subjects can use the client device to nominate authorised users. In one example, to achieve this the one or more processing devices maintains a list of potential authorised users, such as registered healthcare professionals. The client device can then retrieve an indication of one or more authorised users from the one or more processing devices, display a list of the one or more authorised users and then determine selection of one or more of the authorised users in accordance with subject input commands This allows a subject to specify those authorised users that are able to view their subject data, allowing this to be used by the one or more processing devices to maintain the index.

In order for retrieved subject data to be provided to authorised users, the one or more processing devices receives a subject data request from a user client device and then selectively provides the retrieved subject data in response to the subject data request. In particular, the one or more processing devices determines an identity of a user from the subject data request, determines if the user is a selected authorised user and then selectively provides retrieved subject data to the user client device in response to a positive determination. Furthermore, the one or more processing devices can determine an access permission of the user, and then select subject data to be retrieved based on the access permission. This allows the amount or level of detail of the retrieved subject data to be tailored for different users depending on their access permissions.

Such subject data requests can be provided on a one on one basis, so that the request relates to data for a single subject. Alternatively, this can be used to query subject data relating to a group of subjects for example in order to analyse subject data for research purposes. In this latter case, the one or more processing devices receives a subject data request from a user client device relating to a group of one or more subjects. The one or more subjects are typically defined based on parameters, such as physical characteristics, particular wellness or disease states, or the like. The one or more processing devices then generates a query to query subject data stored in the one or more subject databases and retrieves subject data from the one or more subject databases using the query. The one or more processing devices then applies a de-identification algorithm to the retrieved the subject data, providing the de-identified subject data to the user client device. The de-identification can be performed in any suitable manner, such as by using a k-anonymization algorithm, or the like. This allows subject data for multiple subjects to be mined for research purposes or the like, whilst ensuring the required level of privacy is maintained.

To further protect the subject data, the subject data is typically stored in the one or more subject databases in an encrypted form. In this example the one or more processing devices includes an encryption module that encrypts subject data as it is stored in the one or more subject databases and decrypts retrieved subject data that is extracted from the one or more subject databases. Additionally, communication between the one or more processing devices and the client device is generally encrypted using a suitable encryption technique. Such encryption can involve using a session key for each communication session between the client device and processing device and/or a private public key pair associated with the one or more processing devices and/or a private public key pair associated with the client device. It will be appreciated that other secure transfer techniques could also be used depending on the preferred implementation.

In addition to simply displaying a body status indicator, the system can also be adapted to compare a body status indicator or measured body parameter value to notification criteria and then selectively generate a notification in response to results of the comparison. In particular, notification criteria typically include either a body status indicator or body parameter value, or a change in a body status indicator or body parameter value, falling outside a reference range, which can be indicative of an adverse wellness state. This can be utilised in order to notify the subject, and/or authorised users such as healthcare professionals, in the event that there is a health issue. For example, if a heart problem is detected, the system can automatically notify a healthcare professional, allowing them to contact the user, for example to schedule a consultation.

The notification could be of any suitable form and could include a simple message, such as an email, SMS, or the like, optionally including additional information, such as an indication of the relevant body status indicator and/or body parameter value, historical values or the like. Additionally, this could be used to trigger further communication, for example launching a video chat application on the client device, to allow a remote consultation to be performed. It will be appreciated that this provides a mechanism to initiate further action in the event that a healthcare issue is detected.

In addition to simply determining measurement data, in one example the client device is adapted to seek further information from the subject, for example information regarding symptoms currently suffered by the subject, physical characteristics of the user, or the like. This can be achieved by having the client device display a question to the subject and determines an indication of at least one response in accordance with subject input commands The response can then be provided as part of the collected subject data to the processing system for storage. This enables a wide range of different information to be collected from the subject, whilst performing this by displaying only one or two questions each time the device is used allows this to be performed in a non-intrusive manner.

The client device can also be adapted to communicate with the measuring device to control the measurement procedure that is to be performed. This can be performed in response to successful authentication of the subject, so that commencement of the measurement only happens once the subject is authenticated, although other triggers could be used. It will also be appreciated however that as an alternative, a standard measurement process could be performed with results being analysed depending upon body status indicators to be displayed.

To allow suitable communication with the measuring device as well as the one or more processing devices, the client device typically includes a first wireless communications module that allows the client device to communicate with the measurement device and a second wireless communications module that allows the client device to communicate with the remote processing device via the communications network. This can be used to allow a short range wireless communications protocol, such as Bluetooth to be used for communication with the measuring device, thereby reducing the chance of third parties intercepting measurement data, whilst an alternative communications channel, such as a Wi-Fi or cellular communication is used for communication with the one or more processing devices. In one example, the client device is a smart phone or tablet, although it will be appreciated that other devices could be used.

Typically, the measuring device includes a plurality of electrodes in electrical contact with the subject, at least one sensor coupled to a number of the plurality of electrodes, at least one sensor being adapted to measure at least one body signal in the subject via the plurality of electrodes and a measuring device that receives an indication of at least one body parameter value using the at least one body signal. The measuring device may also include a signal generator coupled to a first subset of electrodes to generate a drive signal which is applied to the subject via the first subset of electrodes with the sensor being coupled to a second subset of electrodes allowing impedance measurements to be performed. This arrangement allows a variety of measurement data to be collected including the values relating to respiration parameters, such as a breathing rate, cardiac parameters, such as a heart rate, blood pressure or the like, and impedance parameters, such as value indicative of intracellular and/or extracellular fluids, or the like.

Once collected, the measurement data can be transferred to the client device, allowing this to be partially analysed to determine either a measured body parameter value or a body status indicator, such as an indication of relative fluid levels, abnormal fluid levels, body composition parameters, or the like, before the collected subject data is provided to the one or more processing devices for storage.

In one example, the system includes one or more first processing devices, that are typically provided at a clinician, or other healthcare provider, and more typically form part of the clinician or healthcare providers internal computer systems. The system also typically includes one or more second processing devices, that are typically provided remotely to the clinician, or other healthcare provider, and more typically adapted to interface with first processing devices provided at multiple different healthcare providers to enable consolidation of subject data.

In this instance, the one or more first processing devices determine identity information indicative of an identity of the subject, retrieve an identifier from an index at least in part using the identity information, the index being indicative of a respective identifier associated with each of a plurality of subjects and store the collected subject data in one or more first subject databases to allow stored subject data to be used in calculating one or more body status indicators indicative of a body status of the subject. Thus, the clinicians can retain a copy of subject data for each of the patients for which they are responsible.

Meanwhile, the one or more second processing devices receive at least some subject data and a respective identifier for at least one subject from the one or more first processing systems and store the subject data in one or more second subject databases in accordance with the respective identifier, to thereby allowing subject data from a plurality of subjects to be analysed. Thus, this allows subject data to be collected from multiple clinicians and analysed collectively, allowing the analysis to be performed on subject data collected from a wider range of individuals.

A further benefit of the above described arrangement is that the second processing devices only ever receive subject data and an identifier, and absent of the index accessible to the first processing devices is unable to identify the particular subject to which the subject data relates. In other words, the subject data is de-identified in the second processing devices and associated second databases.

In contrast data stored in the first subject database(s) is not de-identified in the sense that the first processing system has access to the index and hence can be used to identify the subject. In one example, the subject data is stored in the one or more first databases with the respective identifier, meaning the subject data within the database is coded, in the sense that the subject data itself does not identify the subject, but is linked to an identifier, which can be used by the first processing devices to identify the subject using the index.

It will therefore be appreciated that the above described processes, address the technical challenge of consolidating subject data from a significant number of sources, whilst ensuring the subject data is appropriately de-identified, whilst also allowing for subsequent analysis of the subject data as will be described in more detail below.

In one example, the client device provides the identity information to the one or more first processing systems, the identity information including authentication information supplied by the subject and wherein the one or more first processing devices authenticate the subject using the authentication information to determine the identity of the subject. Thus, in this example, the first processing device(s) authenticate the subject, and hence are the only hardware part of the system that is ever in possession of information that can identify the subject. However, it will be appreciated that alternatively, the client device can authenticate the subject using authentication information supplied by the subject and provides identity information in the form of an indication of the identity of the subject to the one or more first processing systems in response to authentication of the subject.

In one example, the client device transfers identity information and subject data separately so that if communications between the client device and first processing systems are intercepted, this would not allow both the identity and subject data to be determined. To achieve this, the client device transfers identity information to the one or more first processing devices, receives the respective identifier from the one or more first processing devices and transfers the subject data together with the respective identifier to the one or more first processing devices. As part of this, the client device can execute a client device software application that enables encrypted communication with a server application executed by the one or more first processing devices. Furthermore, different encryption keys, such as different session keys could be used when transferring the identity information and the subject data, again to reduce the likelihood of third parties being able to identify the subject associated with the subject data.

In one example, the one or more processing devices analyse the subject data by performing machine learning using at least subject data for each of a plurality of subjects in order to derive models that can be used in determining body status indicators from measurement data.

In one particular example, this is achieved by one or more third processing devices, which retrieve subject data and respective subject identifiers from the one or more second processing devices for each of a number of subjects, use the respective subject identifiers to retrieve healthcare data for each of a number of subjects from the one or more first processing devices and analyse the subject data and healthcare data to derive one or more models for use in determining body status indicators from measurement data. The use of the third processing devices allows the analysis to be performed by third parties that specialise in which analysis, whilst still maintaining privacy of the data.

As part of the above process, the first processing systems can retrieve healthcare data by interfacing with an electronic healthcare record system to retrieve healthcare data relating to a respective subject or by retrieving this from the one or more first subject databases. In this regard, the subject data can further include an indication of at least one physical characteristic, an indication of at least one symptom and at least one body status indicator, allowing these to be used as part of the analysis.

In one example, the client device, presents a user interface to collect authentication information, an indication of at least one physical characteristic or an indication of at least one symptom. This can be achieved using a graphical interface, and/or specific hardware, such as a biometric reader for determining the authentication information. The client device also typically includes a display that displays at least one body status indicator and/or measured body parameter values.

The one or more processing devices are generally adapted to generate unique identifiers for each of the plurality of subjects. This can be performed centrally by the second processing devices, but more typically is performed collectively, for example by having the second processing devices assign a respective portion of the identifier to each of a number healthcare providers, with the first processing devices then being used internally to generate a respective identifier for each subject that is a patient of the healthcare provider.

The one or more first processing devices can periodically upload at least some of the subject data to the one or more second processing devices for storage in the one or more second databases. This allows batch uploads to be performed, which in turn reduces the burden on the infrastructure of both the healthcare provider and the entity hosting the subject data for multiple healthcare providers.

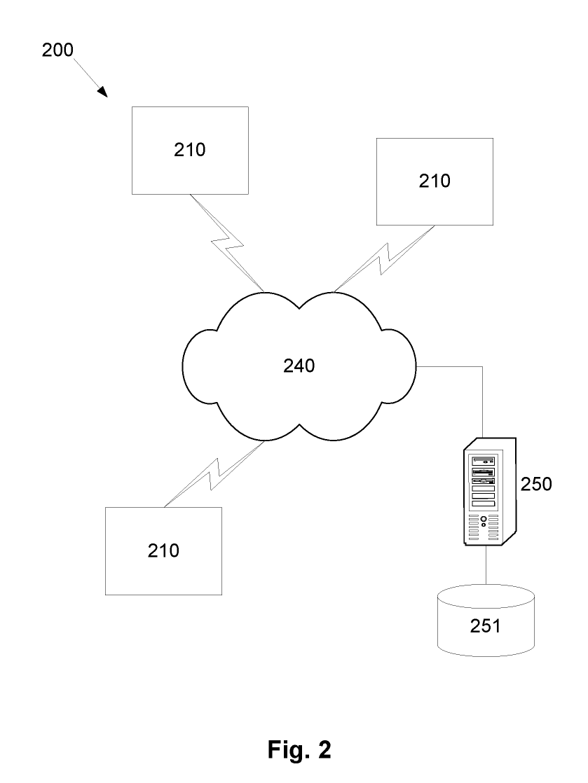

A specific example system will now be described in more detail with reference to FIGS. 2 to 5.

In this example, the system 200 includes a number of measuring systems 210 coupled via a communications network 240 to one or more other measuring systems 210 and/or one or more processing devices, such as a server 250, which may in turn be coupled to a database 251. This arrangement allows subject data to be collected by the measurement systems 210 and provided to the server 250 for storage and optional analysis. Collected subject data may also be stored in the database 251 together with other information, such as body state indicators, allowing this information to be remotely accessed and viewed by authorised users, such as clinicians, or the like.

In the above arrangement, the communications network 240 can be of any appropriate form, such as the Internet and/or a number of local area networks (LANs) and provides connectivity between the measuring systems 210 and the server 250. It will however be appreciated that this configuration is for the purpose of example only, and in practice the measuring systems 210 and server 250 can communicate via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, or the like.

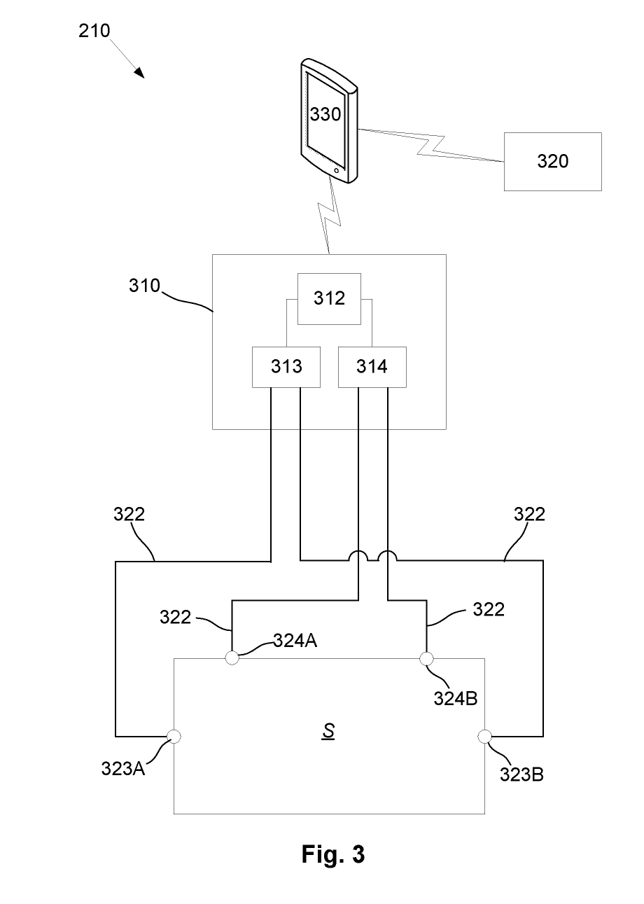

An example measuring system will now be described in further detail with reference to FIG. 3.

In this example, the measuring system includes an impedance measuring unit having an impedance measuring device 310, which is in turn in communication with a processing system in the form of a client device 330, such as a portable computer system, mobile phone, tablet or the like. One or more optional physical characteristic sensors 320 can also be provided for capturing information regarding physical characteristics of an individual/subject.

The nature of the physical characteristic sensors 320 will vary depending on the characteristics to be measured, and could include for example an image capture device, such as a camera, body scanner, DEXA (Dual-Energy X-ray Absorptiometry), 3D laser or optical scanning, or the like, for measuring a height and/or body segment dimensions. Additionally or alternatively, this could include electronic scales for measuring a weight, and other monitoring equipment, for example for measuring heart rate, blood pressure or other characteristics.

The impedance measuring device 310 typically includes a measuring device processor 312 coupled to at least one signal generator 313 and at least one sensor 314, which are in turn coupled to respective drive and sense electrodes 323A, 323B and 324A, 324B, via leads 322. In use, the signal generator 313 generates a drive signal, which is applied to the individual/subject S via the drive electrodes 323A, 323B, whilst the sensor 314 measures a response signal via the sense electrodes 324A, 324B. In use, the measuring device processor 312 controls the at least one signal generator 313 and the at least one sensor 314, allowing the impedance measurements to be performed.

In particular, the measuring device processor 312 is adapted to generate control signals, which cause the signal generator 313 to generate one or more alternating signals, such as voltage or current signals of an appropriate waveform, which can be applied to a subject S, via the first electrodes 323A, 323B and processing received signals from the sensor 314. It will be appreciated that the measuring device processor 312 may be any form of electronic processing device capable of performing appropriate control, and could include an FPGA (field programmable gate array), or a combination of a programmed computer system and specialised hardware, or the like.

The signal generator 313 could be of any appropriate form, but will typically include digital to analogue converters (DACs) for converting digital signals from the one or more processing devices to analogue signals, which are amplified to generate the required drive signals, whilst the sensor 314 typically includes one or more amplifiers for amplifying sensed response signals and analogue to digital converters (ADCs) to digitise the analogue response signals and providing digitised response signals to the one or more processing devices.

The nature of the alternating drive signal will vary depending on the nature of the measuring device and the subsequent analysis being performed. For example, the system can use Bioimpedance Analysis (BIA) in which a single low frequency signal is injected into the subject S, with the measured impedance being used directly in the determination of biological parameters. In one example, the applied signal has a relatively low frequency, such as below 100 kHz, more typically below 50 kHz and more preferably below 10 kHz. In this instance, such low frequency signals can be used as an estimate of the impedance at zero applied frequency, commonly referred to as the impedance parameter value R₀, which is in turn indicative of extracellular fluid levels.

Alternatively, the applied signal can have a relatively high frequency, such as above 200 kHz, and more typically above 500 kHz, or 1000 kHz. In this instance, such high frequency signals can be used as an estimate of the impedance at infinite applied frequency, commonly referred to as the impedance parameter value R_{∞}, which is in turn indicative of a combination of the extracellular and intracellular fluid levels, as will be described in more detail below.

Alternatively and/or additionally, the system can use Bioimpedance Spectroscopy (BIS) in which impedance measurements are performed at each of a number of frequencies ranging from very low frequencies (1 kHz and more typically 3 kHz) to higher frequencies (1000 kHz), and can use as many as 256 or more different frequencies within this range. Such measurements can be performed by applying a signal which is a superposition of plurality of frequencies simultaneously, or a number of alternating signals at different frequencies sequentially, depending on the preferred implementation. The frequency or frequency range of the applied signals may also depend on the analysis being performed.

When impedance measurements are made at multiple frequencies, these can be used to derive one or more impedance parameter values, such as values of R₀, Zₒ, R_{∞}, which correspond to the impedance at zero, characteristic and infinite frequencies. These can in turn be used to determine information regarding both intracellular and extracellular fluid levels, as will be described in more detail below.

A further alternative is for the system to use Multiple Frequency Bioimpedance Analysis (MFBIA) in which multiple signals, each having a respective frequency are injected into the subject S, with the measured impedances being used in the assessment of fluid levels. In one example, four frequencies can be used, with the resulting impedance measurements at each frequency being used to derive impedance parameter values, for example by fitting the measured impedance values to a Cole model, as will be described in more detail below. Alternatively, the impedance measurements at each frequency may be used individually or in combination.

Thus, the measuring device 310 may either apply an alternating signal at a single frequency, at a plurality of frequencies simultaneously, or a number of alternating signals at different frequencies sequentially, depending on the preferred implementation. The frequency or frequency range of the applied signals may also depend on the analysis being performed.

In one example, the applied signal is generated by a voltage generator, which applies an alternating voltage to the subject S, although alternatively current signals may be applied. In one example, the voltage source is typically symmetrically arranged, with two signal generators 313 being independently controllable, to allow the signal voltage across the subject to be varied, for example to minimise a common mode signal and hence substantially eliminate any imbalance as described in copending patent application number WO2009059351.

As the drive signals are applied to the subject, the sensor 314 then determines the response signal in the form of the voltage across or current through the subject S, using second electrodes 324A, 324B. Thus, a voltage difference and/or current is measured between the second electrodes 324A, 324B. In one example, a voltage is measured differentially, meaning that two sensors 314 are used, with each sensor 314 being used to measure the voltage at each second electrode 324A, 324B and therefore need only measure half of the voltage as compared to a single ended system. Digitised response signals are then provided to the measuring device processor 312, which determines an indication of the applied drive signal and measured response signals, and optionally uses this information to determine measured impedances.

In the above arrangement, four electrodes are shown, with two forming drive electrodes and two forming sense electrodes. However, this is not essential, and any suitable number of electrodes could be used. In one preferred arrangement, four drive and four sense electrodes are provided, with these being placed in contact with the hands and feet, allowing for whole of body and segmental impedance measurements to be performed. Furthermore, although a single signal generator and sensor are shown, a respective signal generator and sensor could be used for each drive and sense electrode, respectively, and the described arrangement is for the purpose of illustration only.

Additionally, the electrodes and sensors could be used passively in absence of any applied current signal in order to detect electrical signals, such as ECG signals or the like.

In the above arrangement, the client device 330 is coupled to the measuring device processor 312, allowing operation of the impedance measuring device to be controlled. In particular, the client device 330 can be used to instruct the measuring device processor 312 on a particular sequence of impedance measurements that need to be performed, further receiving either an indication of the drive/sense signals and/or measured impedance values. The client device 330 can then optionally perform further processing, for example to determine the impedance indicators, although alternatively this may not be required and raw impedance data could be provided to the server 250 for analysis.

The client device 330 can also combine impedance values or indicators with information regarding indications of disease states and physical characteristics determined either by manual user input or based on signals from one or more physical characteristic sensors. This allows the client device 330 to generate the collected subject data, which is then transferred via the communications network 240 to the server 250. However, alternatively, the server 250 could obtain the indication of disease states and/or physical characteristic from other data sources, depending on the preferred implementation.

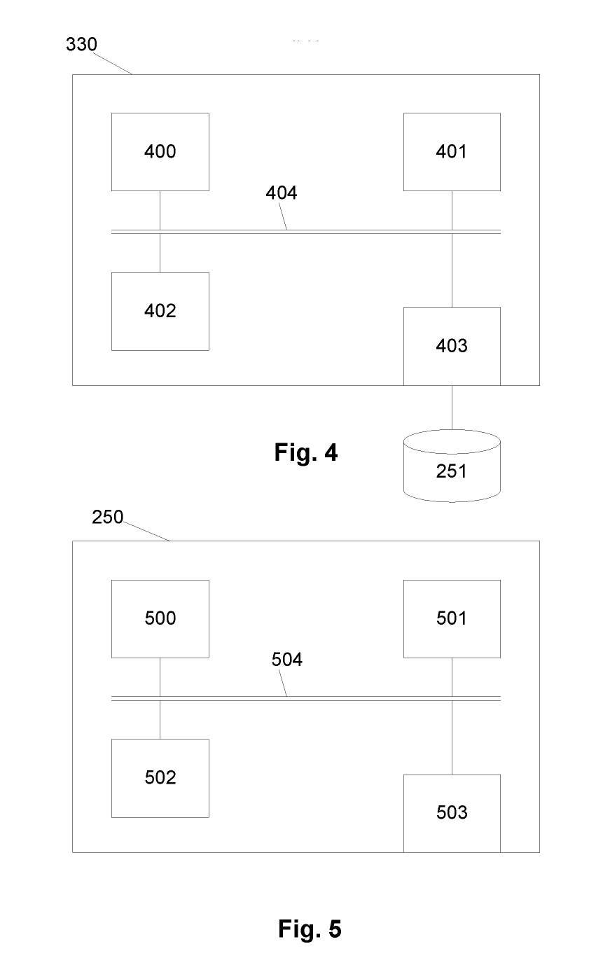

Accordingly, it will be appreciated that the client device 330 can be of any appropriate form and one example is shown in FIG. 4. In this example, the client device 330 includes at least one microprocessor 400, a memory 401, an input/output device 402, such as a keyboard and/or display, and an external interface 403, interconnected via a bus 404 as shown. The external interface 403 can be utilised for connecting the client device 330 to peripheral devices, such as the communications networks 240, databases, other storage devices, or the like. Although a single external interface 403 is shown, this is for the purpose of example only, and in practice multiple interfaces using various methods (e.g. Ethernet, serial, USB, wireless or the like) may be provided.

In use, the microprocessor 400 executes instructions in the form of applications software stored in the memory 401 to allow communication with the server 250, for example to allow reference data to be provided to the sever, or the like.

Accordingly, it will be appreciated that the client device 330 may be formed from any suitable processing system, such as a suitably programmed PC, Internet terminal, lap-top, or hand-held PC, and in one preferred example is either a tablet, or smart phone, or the like. Thus, in one example, the client device 330 is a standard processing system such as an Intel Architecture based processing system, which executes software applications stored on non-volatile (e.g., hard disk) storage, although this is not essential. However, it will also be understood that the client devices 330 can be any electronic processing device such as a microprocessor, microchip processor, logic gate configuration, firmware optionally associated with implementing logic such as an FPGA (Field Programmable Gate Array), or any other electronic device, system or arrangement.

An example of a suitable server 250 is shown in FIG. 5. In this example, the server includes at least one microprocessor 500, a memory 501, an optional input/output device 502, such as a keyboard and/or display, and an external interface 503, interconnected via a bus 504 as shown. In this example the external interface 503 can be utilised for connecting the server 250 to peripheral devices, such as the communications networks 240, databases 251, other storage devices, or the like. Although a single external interface 503 is shown, this is for the purpose of example only, and in practice multiple interfaces using various methods (e.g. Ethernet, serial, USB, wireless or the like) may be provided.

In use, the microprocessor 500 executes instructions in the form of applications software stored in the memory 501 to allow the required processes to be performed, including communicating with the client devices 330, and optionally receiving, analysing and/or displaying results of impedance measurements. The applications software may include one or more software modules, and may be executed in a suitable execution environment, such as an operating system environment, or the like.

Accordingly, it will be appreciated that the server 250 may be formed from any suitable processing system, such as a suitably programmed client device, PC, web server, network server, or the like. In one particular example, the server 250 is a standard processing system such as an Intel Architecture based processing system, which executes software applications stored on non-volatile (e.g., hard disk) storage, although this is not essential. However, it will also be understood that the processing system could be any electronic processing device such as a microprocessor, microchip processor, logic gate configuration, firmware optionally associated with implementing logic such as an FPGA (Field Programmable Gate Array), or any other electronic device, system or arrangement. Accordingly, whilst the term server is used, this is for the purpose of example only and is not intended to be limiting.

Whilst the server 250 is a shown as a single entity, it will be appreciated that the server 250 can be distributed over a number of geographically separate locations, for example by using processing systems and/or databases 251 that are provided as part of a cloud based environment. Thus, the above described arrangement is not essential and other suitable configurations could be used.

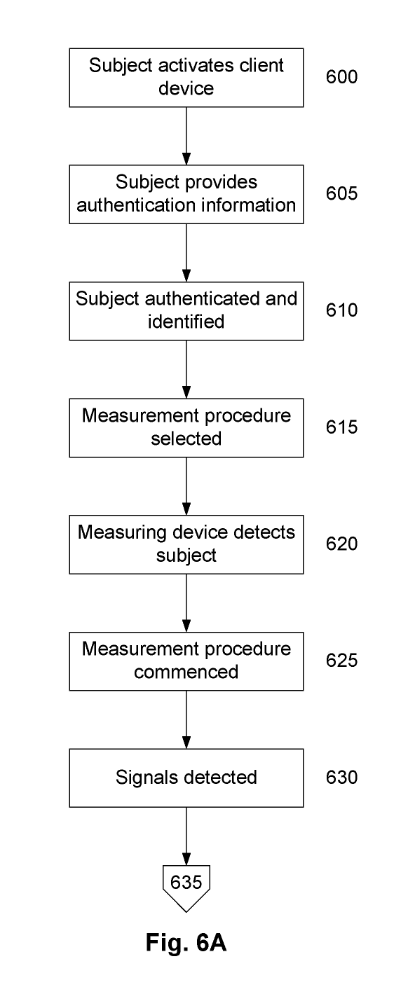

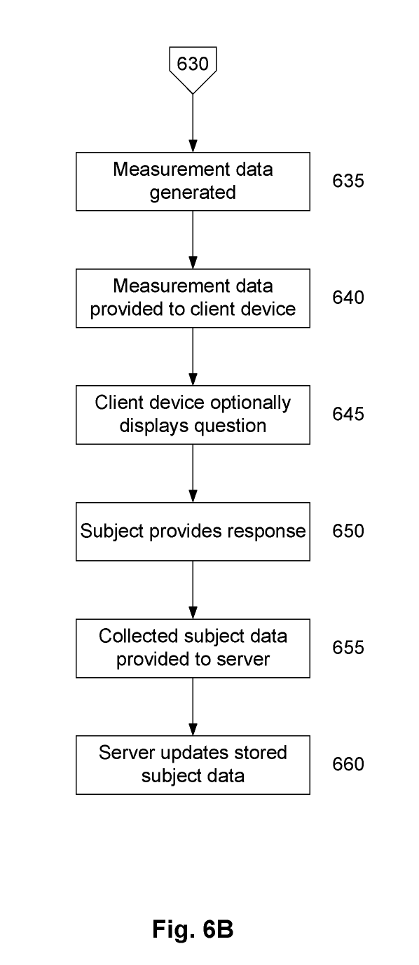

Operation of the system will now be described in further detail with reference to FIGS. 6A and 6B.

For the purpose of these examples it will also be assumed that subjects use the client devices 330 control the measuring device 310 and any characteristics sensors, allowing impedance and/or other measurements to be performed and allowing information regarding physical characteristics to be collected. This is typically achieved by having the subject interact with the system via a GUI (Graphical User Interface), or the like presented on the client device 330, which may be generated by a local application, or hosted by the server 250, which is typically part of a cloud based environment, and displayed via a suitable application, such as a browser or the like, executed by the client device 330. Actions performed by the client device 330 are typically performed by the processor 400 in accordance with instructions stored as applications software in the memory 401 and/or input commands received from a user via the I/O device 402. Similarly, actions performed by the server 250 are performed by the processor 500 in accordance with instructions stored as applications software in the memory 501 and/or input commands received from a user via the I/O device 502, or commands received from the client device 330.

The system utilises multiple measuring and client devices 310, 330, which interact with one or more central servers 250, typically forming part of a cloud based environment. This allows subject data to be collected from a number of different sources, and then aggregated and stored centrally.

Whilst the following example focuses on the analysis of impedance indicators only, it will be appreciated that the techniques could be extended to include other parameter values, such as other vital signs or the like, and reference to impedance indicators only is not intended to be limiting.

However, it will be appreciated that the above described configuration assumed for the purpose of the following examples is not essential, and numerous other configurations may be used. It will also be appreciated that the partitioning of functionality between the measuring device 310, client devices 330, and servers 250 may vary, depending on the particular implementation.

In this example, at step 600 the subject activates the client device 330. This would typically involve opening an application installed on the client device in order to allow the measurement process to commence. At step 605 the subject is prompted to provide authentication information, which may involve supplying biometric information, for example by performing an iris scan, fingerprint scan, or the like, or alternatively entering information such as a password, PIN or similar. As a further alternative, authentication information can be derived from the results of measurements performed below, for example by basing authentication information on a combination of measured parameters, such as a height, weight, and impedance values.

Having provided the authentication information at step 610, the subject is authenticated and identified. The authentication process can be performed locally by the client device 330, for example by comparing received authentication information to previously stored authentication information in the client device memory 401. Alternatively this may be performed remotely by the server 250 by having the client device 330 forward the authentication information to the server 250, and having the server 250 compare this to reference authentication information stored in the one or more subject databases.

At step 615 a measurement procedure is selected. In this regard a number of different measurement procedures may be implemented depending on a range of factors, such as the software modules loaded onto the client device 330, conditions suffered by the subject, body status indicators to be displayed, or the like. The process of selecting a measurement procedure can involve displaying information regarding available measurement procedures allowing a user to select one of these. Alternatively, this may be performed automatically, for example by selecting a measurement procedure based on the software installed on the device. As a further alternative this may not be required if identical measurement processes are used irrespective of the information presented to the subject.

At step 620 the measurement device 310 detects the subject. In this regard, the subject will generally place their hands and/or feet in contact with electrodes, with this being detected by the measuring device 310 thereby causing the measurement procedure to commence at step 625.

The nature of the measurement procedure will vary depending upon the preferred implementation. In one example, a sequence of current signals are applied to the body with resulting voltage signals being measured in impedance parameter values relating to the body to be determined.

In this case, the measuring device processor 312 controls the signal generator and sensor, causing the drive signals to be applied to the subject and causing the corresponding response signals to be measured, allowing the measuring device processor 312 to determine both the drive and response signals.

The response signal will be a superposition of voltages generated by the human body, such as the ECG (electrocardiogram), voltages generated by the applied signal, and other signals caused by environmental electromagnetic interference. Accordingly, filtering or other suitable analysis may be employed to remove unwanted components.

The acquired signal is typically demodulated to obtain the impedance of the system at the applied frequencies. One suitable method for demodulation of superposed frequencies is to use a Fast Fourier Transform (FFT) algorithm to transform the time domain data to the frequency domain. This is typically used when the applied current signal is a superposition of applied frequencies. Another technique not requiring windowing of the measured signal is a sliding window FFT.

In the event that the applied current signals are formed from a sweep of different frequencies, then it is more typical to use a signal processing technique such as multiplying the measured signal with a reference sine wave and cosine wave derived from the signal generator, or with measured sine and cosine waves, and integrating over a whole number of cycles. This process, known variously as quadrature demodulation or synchronous detection, rejects all uncorrelated or asynchronous signals and significantly reduces random noise. Other suitable digital and analogue demodulation techniques will be known to persons skilled in the field.

Alternatively passive sensing can be performed in order to detect ECG or other similar signals.

Irrespective of the mode of sensing used at step 630 signals are detected with these being used by the measurement device 310 to generate measurement data. The measurement data can include raw data or may include partially or fully processed data.

For example, in the case of BIS, impedance or admittance measurements are determined from the signals at each frequency by comparing the recorded voltage and the current through the subject. The demodulation algorithm can then produce amplitude and phase signals at each frequency, allowing an impedance value at each frequency to be determined. Whilst the measured impedance can be used directly, in one example, the measured impedance is used to derive an impedance parameter, and in particular an impedance (resistance) at zero frequency, R₀, equals the extracellular resistance Rₑ.

For example, minimal processing such as filtering of signals is typically performed by the measuring device. Additionally, voltage and current signals may be processed in order to determine impedance values such as resistance, reactance and phase angle values. The measurement data can include processed signals as well as raw data depending on the preferred implementation. In general inclusion of the raw data is preferred as this can allow data to be reprocessed at a later date, for example allowing this to be analysed using improved algorithms

At step 640 the measurement data is provided to the client device 330, typically using a short range wireless communication protocol such as Bluetooth, NFC or the like. The use of a short range protocol reduces the likelihood of the measurement data being intercepted by third parties. Irrespective of this however the measurement data can be encrypted utilising a public key of the client device. With the data also further being optionally signed by a private key of the measuring device to thereby verify the source of the measurement data and hence help ensure measurement data integrity.

At step 645 the client device 330 optionally displays a question to the subject. The question may relate to symptoms or other information required by the system, such as information regarding physical characteristics, exercise, diet or the like, allowing additional information regarding the subject to be collected. By doing this each time a measurement is performed allows a wide range of data regarding the subject to be collected, without placing an undue burden on the subject. At step 650 the subject provides a response, for example by selecting an appropriate input option with this being used by the client device 330 to provide collected subject data to the server 250 at step 655.

The collected subject data typically includes the measurement data and any additional data, such as responses to questions, data collected from additional sensors, such as physical characteristic data, but may also include other data such as environmental data, including but not limited to location data, temperature data, of the like.

At step 660 the server 250 updates the subject data by adding the collected subject data. It will be appreciated that this allows subject data relating to the individual to be collected over time, which in turn enables a comprehensive health record to be established directly from measurement data recorded from a measuring device. Once subject data has been recorded, this can be used to generate a body status indicator which is displayed to the subject. The body status indicator can be displayed at any time during the process and this does not need to wait until collected subject data has been uploaded to the server. Indeed, this can be performed concurrently with the data collection process.

The nature of the body status indicator will vary depending on the preferred implementation and the nature of the measurement data. The body status indicator could include a simple recorded value but more typically examined changes in parameter values such as changes in fluid levels or the like. The nature of the body status indicator is not important for the purposes of the current example and numerous body status indicators will be known to those in the art.

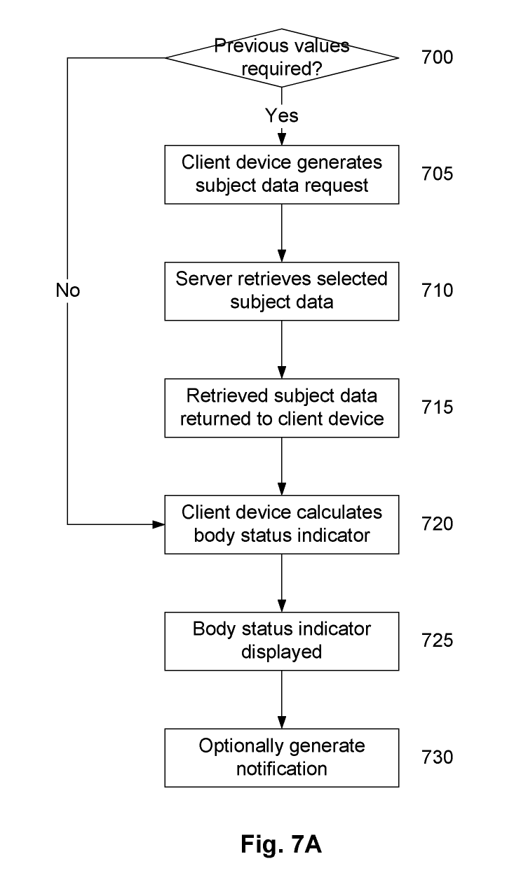

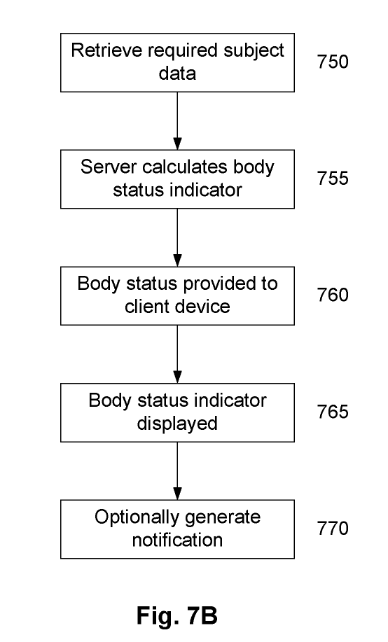

In one example the body status indicator is generated locally using the client device 330, as shown in FIG. 7A.

In this example, at step 700 the client device 330 determines if previously measured body parameter values are required. It will be appreciated that these may not be required, for example if they are already stored locally on the client device 330, or if they are not needed to generate the body status indicator. Assuming previous body parameter values are required, at step 705 the client device 330 generates a subject data request which is transferred to the server 250. At step 710 the server 250 retrieves relevant subject data, returning this retrieved subject data to the client device 330 at step 715. At step 720, the client device 330 calculates the body status indicator, for example by determining a change in the body parameter value, causing this to be displayed to the subject at step 725.

At this stage, the client device 330 may also optionally generate a notification, for example based on comparison of the body parameter value and/or body status indicator to a reference range or other notification criteria. The notification can be displayed on the client device 330, and may include a motivational message, alert, warning, or the like. For example, if the user has an unexpectedly high heart rate, or if fluid levels have changed dramatically in a short period of time, a warning may be displayed to the subject directing them to seek medical attention.

Additionally, and/or alternatively notifications can be provided to other authorised users. As will be described in more detail below, subjects can grant specific users, such as medical practitioners, authorisation to access their subject data. In this instance the client device 330 can generate a notification and transfer this to an authorised user, such as the subject's doctor, alerting them to a particular event. This can be used to allow the medical practitioner to contact the subject directing them to seek medical attention.

It will be appreciated that the above-described process may require that subject data is retrieved and provided to the client device 330. As an alternative to this however the body status indicator could be generated by the server 250 and transferred to the client device, and an example of this will now be described with reference to FIG. 7B.

In this example, the server 250 retrieves required subject data at step 750 and then calculates the body status indicator at step 755. The body status indicator is then transferred to the client device 330 at step 760, allowing this to be displayed to the subject at step 765. The server 250 may then optionally generate a notification at step 770 allowing this to be provided to the client device 330 or a client device of an authorised user as required.

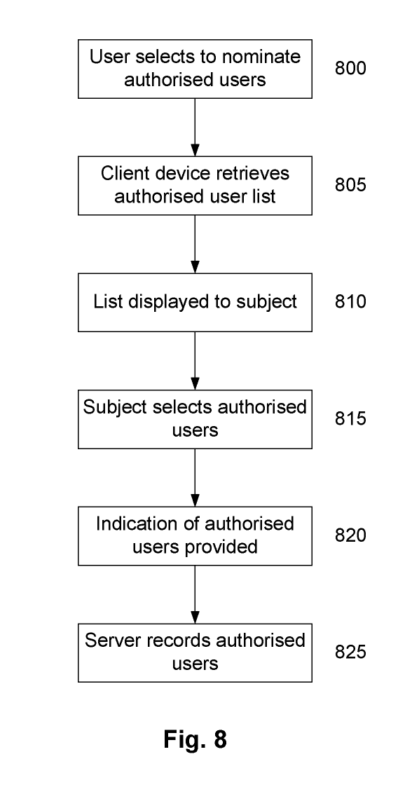

As previously mentioned, the system can be utilised in order to provide authorised users access to the subject's subject data. In order to achieve this, it is typically necessary for the user to designate authorised users able to view their subject data, and an example of this process will now be described with reference to FIG. 8.

In this example, at step 800 the user selects to nominate authorised users, for example utilising a suitable option displayed by the application installed on the client device 330. At step 805 the client device retrieves an authorised user list from the server 250. In this regard the server 250 will maintain a list of registered healthcare professionals, or other individuals, that are able to use the system to view subject data.

At step 810 a list is displayed to the subject allowing the user to select an authorised user from the list at step 815. It will be appreciated that as part of this process, users may enter full or partial details of relevant healthcare professionals, allowing the list to be searched to thereby more rapidly identify relevant authorised users.

An indication of selected authorised users can then be provided to the server 250 at step 820, with the server 250 operating to record authorised users for the respective subject in an index, such as a master patient index.

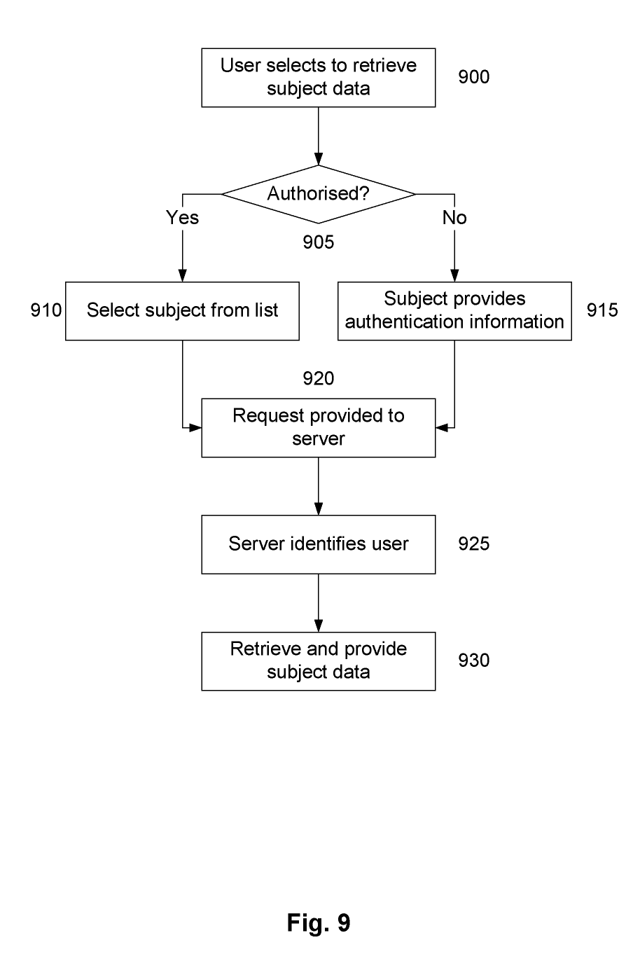

An example of the process for allowing a user to view subject data from another individual will now be described with reference to FIG. 9.

In this example, at step 900 a user selects to retrieve subject data for example by selecting an appropriate input option using the application on a user client device 330.

The next stage in the process will depend on whether the user is already authorised to view the subject's subject data. If so, the user will be presented with a list of subjects for which they are authorised, with this being provided by the server 250, allowing the user to simply select the relevant subject from the list.

However, if the user is not authorised, then the subject can be authenticated in order to provide access to the subject data. To achieve this, the subject is required to provide authentication information via the client device 330. For example, in a hospital environment if a medical practitioner is seeing a new patient they would not typically have authorisation to view that patient's records. Accordingly, the medical practitioner can select the retrieve subject data option and then simply hand their client device 330 to the patient, allowing the patient to provide their authentication information at step 915, by scanning a fingerprint, entering a PIN or password, or the like.

In either case, a subject data request is provided to the server 250 at step 920, with this either including the identity of the subject, or the subject's authentication information, as appropriate. At step 925 the server 250 identifies and optionally authenticates the subject and retrieves and provides the relevant subject data at step 930.

Accordingly, it will be appreciated that this provides a mechanism to allow the subject to provide the user with access to their subject data, simply by undergoing authentication by supplying authentication via the client device 330 of the user. As a further alternative, the subject may choose to undergo authentication using their own client device 330, with this being used to download subject data, which is then transferred to the client device 330 of the medical practitioner, for example using NFC or the like.

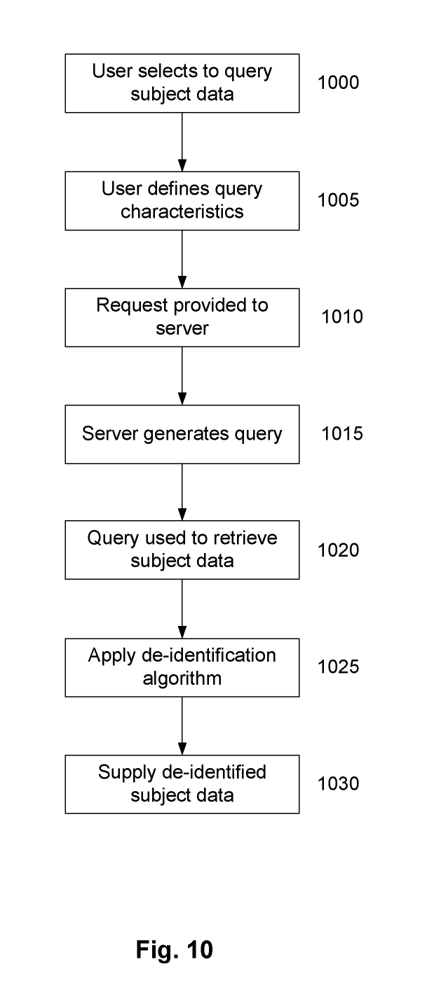

In another example, a user may wish to query subject data, for example to analyse data for multiple subjects. This can be used to allow the data to be mined in order to establish patterns within the data, such as to identify combinations of parameter values that are indicative of a respective disease state. In order to maintain data privacy, it is necessary to ensure that data provided as a result of any such requests are de-identified and an example of this process will now be described with reference to FIG. 10.

In this example, at step 1000, a user selects a query subject data option, for example using an application installed on a client device 330. The user is then typically provided with a prompt or fields, allowing the user to define parameters associated with the query at step 1005. This could include details of disease states or physical characteristics of interest, and is used in order to retrieve subject data relevant to a group of subjects. At step 1010, a query request including the parameters is provided to the server 250, allowing the server to generate a query for querying the one or more subject databases at step 1015. The query is then applied to the database and relevant subject data retrieved at step 1020.

At step 1025 a de-identification algorithm is applied to the retrieved data. The de-identification algorithm is used to remove data that could be used to identify specific individuals. At one level, this would simply include removing identification information such as names and addresses. However, for multi-dimensional datasets, the de-identification process can be more complex, and may require that data is removed, genericised, clustered or the like to avoid individuals being identified. Such de-identification algorithms are known in the art and will not therefore be described in detail. The de-identified retrieved subject data can then be provided to the user at step 1030.

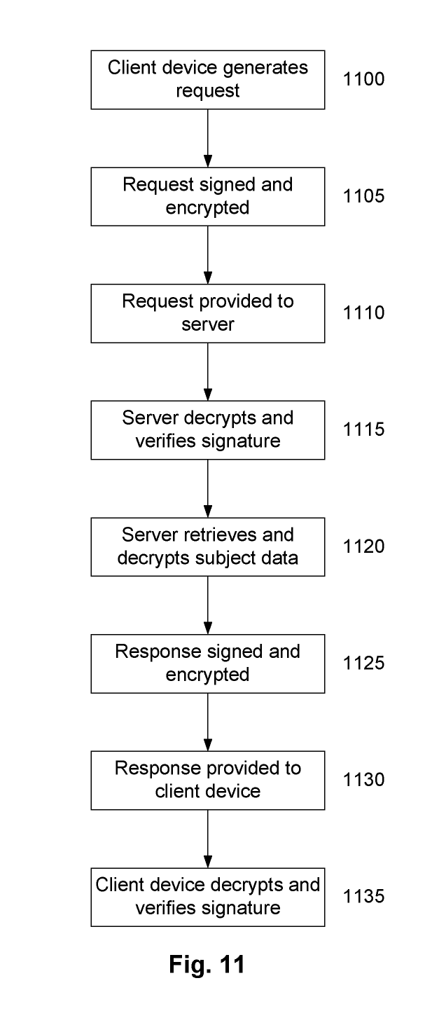

Throughout the above described processes, subject data, including retrieved, collected and stored subject data, is retained in an encrypted form where possible to thereby ensure privacy is maintained. An example of the process for retrieving such data will now be described with reference to FIG. 11.

For the purpose of this example, it is assumed that the server 250 and each client device 330 has a respective public private key pair, and performs encryption using a previously selected asymmetric encryption algorithm.

In this example, at step 1100, a client device 330 generates a request to retrieve subject data. The request is signed using the private key of the client device and encrypted using the public key of the server 250 at step 1105, before being transferred to the server 250 at step 1110. The server 250 is able to decrypt the request using the server private key, and verify the signature using the client device public key. It will be appreciated that this approach allows the server 250 to ensure the request has been provided by a legitimate client device 330, typically previously registered for use with the system, whilst ensuring the request is encrypted, to prevent this being intercepted and spoofed by third parties.

At step 1120, the server 250 queries the one or more subject databases to retrieve the requested subject data. The subject data is stored in an encrypted form, and so as part of this process, the server 250 decrypts the retrieved subject data. At step 1125, the server 250 generates a response including the subject data, with the response being encrypted using the client device public key and signed using the server private key. The response can then be transferred to the client device 330 at step 1130, allowing the client device 330 to decrypt the response using the client device private key and verify the signature using the server public key. This allows the client device 330 to ensure the response has been legitimately created by the server 250, whilst further allowing the data to be decrypted for use as required.

It will be appreciated that a similar approach can be used for transferring collected subject data to the server 250 for storage. These techniques ensure that subject data is encrypted during storage and transfer, thereby ensuring privacy of the subject data is maintained.

Accordingly, it will be appreciated that the above described system provides a mechanism to allow subject data to be collected directly from a measurement device, and integrated into stored subject data, such as an electronic medical record. The stored subject data can be queried and used, for example in interpreting measured body parameter values, as well as to review historical body status records, allowing a healthcare professional and other users to understand changes in body parameter values over time. The system can be adapted to upload raw data, allowing this to be reanalysed as improved analysis techniques are developed. This further assists in allowing data to be mined in order to identify patterns characteristic of certain disease and/or wellness states.

Transfer of measurement and retrieval of subject data is achieved using a client device, such as a subject's mobile phone or tablet. This acts to interface with the measuring device to retrieve and transfer the data, whilst allowing outcomes of measurements to be viewed. This also allows users to be authenticated and identified, using mechanisms such as biometric readers built into the client device, ensuring that collected subject data is correctly stored and retrieved. As part of this, the system can provide an inbuilt mechanism in order to ensure subject data can only be accessed by authorised users, such as medical practitioners, and even then only once authorisation has been provided by the subject themselves. Additionally, the use of a client device such as a phone or tablet, allows this to be used to provide access to medical professionals, as required, for example by way of phone or video calls.

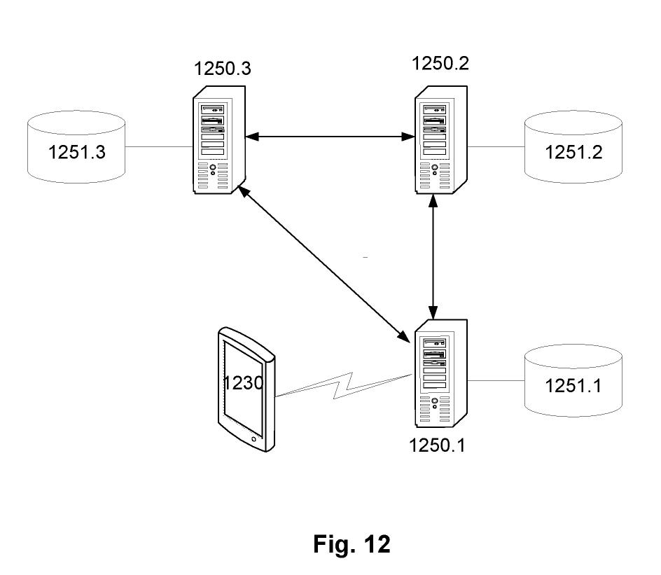

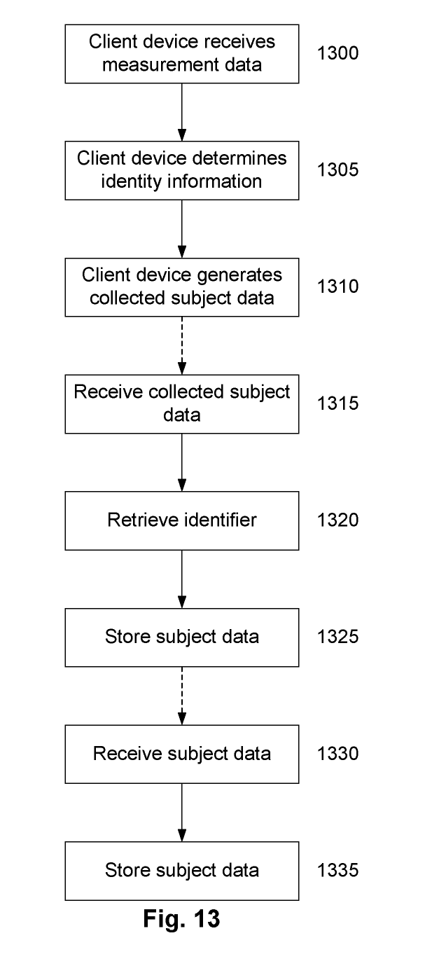

A further example arrangement involving the use of first and second processing devices will now be described with reference to FIGS. 12 and 13.

In this example, the system includes a client device 1230, in communication with a measurement device (not shown in this example) and one or more first processing devices, typically forming part of one or more first processing systems 1250.1, which are in turn connected to one or more first subject databases 1251.1. The first processing devices are in communication with one or more second processing devices, again typically forming part of respective second processing systems 1250.2, connected to second subject databases 1251.2, and optionally in communication with one or more third processing devices, forming part of third processing systems 1250.3, which may be connected to third subject databases 1251.3.

In use, the measuring device acquires measurement data at least partially indicative of impedance measurements performed on a subject, communicating this to the client device 1230, which receives the measurement data at step 1300 and determines identity information indicative of an identity of the subject at step 1310. The identity information could include authentication information, a username and/or password, biometric data or the like, and could be determined as part of a process of authenticating the subject, which may occur prior to or after the measurement is performed.

At step 1310, the client device 1230 then generates collected subject data indicative of the measurement data, measured body parameter values of the subject and/or at least one body status indicator at least partially derived from the measured body parameter values, transferring this and an indication of the identity information to the first processing devices. The identity information could be transferred as part of the subject data, or may be transferred as part of a separate communications step, as will be described in more detail below.

At steps 1315, the first processing device(s) receive the collected subject data and identity information, using the identity information to retrieve an indication of the an identifier from an index at step 1320. In this regard, the index is indicative of a respective identifier associated with each of a plurality of subjects, and typically corresponds to an index of patient specific to a particular healthcare provider, as will be described in more detail below. The collected subject data is then stored in in one or more first subject databases 1251.1 at step 1325, to allow stored subject data to be used in calculating one or more body status indicators indicative of a body status of the subject.

Additionally, the first processing device(s) upload subject data and a respective identifier to the second processing system(s), which receive the subject data and the respective identifier at step 1330, using this to store the subject data in one or more second subject databases 1251.2 in accordance with the respective identifier at step 1335.

Accordingly, it will be appreciated that the above described arrangement allows subject data to be measured using a measuring device, and then uploaded to the first processing devices, via the client device 1230, for storage. In one preferred example, the first processing devices form part of first processing systems 1250.1, such as computer servers or the like, provided at a clinician, or other healthcare provider. This enables the clinician to store and access data relating to measurements, and in particular impedance measurements, performed on patients. The subject data can be de-identified by associating the subject data with a unique identifier, which is associated with the subject using an index retained by the clinician/healthcare provider. The subject data can then be provided in a de-identified form to second processing devices, typically forming part of remote servers, allowing subject data from a range of different clinicians/healthcare providers to be consolidated for analysis, whilst retaining data privacy.

As previously mentioned, the above described processes can address the technical challenge of consolidating subject data from a significant number of sources, whilst ensuring the subject data is appropriately de-identified, whilst also allowing for subsequent analysis of the subject data as will be described in more detail below.

A number of further features will now be described.

In one example, a processing device can determine a body status indicator to be displayed and retrieve at least some of the stored subject data for the subject in accordance with the determined body status indicator. This allows previous measurements stored in either the first or second databases to be retrieved and used in calculating a body status indicator, for example if the body status indicator involves a longitudinal analysis of changes in parameter values compared to previous measurements. The processing device can then generate the body status indicator, providing this to the client device for display, or provide retrieved subject data to the client device allowing the client device to generate the body status indicator. Alternatively, the body status indicator could be displayed by the first processing device, for example displaying this directly to a clinician, or the like.

In one example, the identity information includes authentication information supplied by the subject, for example in the form of biometric data or a password provided via a hardware or software interface. The authentication information can be provided to the one or more first processing devices, allowing the processing devices to authenticate the subject to determine the identity of the subject. Alternatively, the client device can authenticate the subject using authentication information supplied by the subject and provides the identity information in the form of an indication of the identity of the subject in response to authentication of the subject. It will be appreciated that this latter approach relies on the client device having stored authentication information previously provided by the subject, which can be undesirable if the clinician or healthcare provider uses a number of different client devices with different subjects.

In one example, the client device transfers identity information to the one or more first processing devices, which determine the identifier associated with the subject, using the index, and then return this to the client device. The client device receives the respective identifier from the one or more first processing devices and then transfers the collected subject data together with the respective identifier to the one or more first processing devices. This ensures the identity of the subject and the associated subject data are not transmitted at the same time, which in turn can help reduce the likelihood of the subject data being intercepted and associated with the identity of the subject by a third party. To further assist with this, the client device can execute a client device software application that enables encrypted communication with a server application executed by the one or more first processing devices.

Having obtained subject data, the processing device(s) can analyse the subject data by performing machine learning using at least subject data for each of a plurality of subjects in order to derive models that can be used in determining body status indicators from measurement data. Thus, this allows subject data from a range of different subjects can be mined in order to facilitate in interpreting measurements relating to a particular subject.

In one example, this is performed by third processing devices that retrieve at least some of the stored subject data and respective subject identifiers from the one or more second processing devices for each of a number of subjects. The third processing devices then use the respective subject identifiers to retrieve healthcare data for each of a number of subjects, typically from the one or more first processing devices, allowing the retrieved subject data and healthcare data to be analysed to derive one or more models for use in determining body status indicators from measurement data. In this example, the first processing device(s) can retrieve healthcare data by interfacing with an electronic healthcare record system or can retrieve the healthcare data from local or other internal databases. Thus, this avoids the need for the second processing systems to access healthcare data, allowing this to be performed by separate third processing systems in a manner that ensures compliance with privacy requirements.

The subject data can include an indication of at least one physical characteristic, an indication of at least one symptom and/or at least one body status indicator, with this information optionally being collected by the client device, and/or by a clinician, during a consultation.

In one example, the client device can present a user interface to collect the authentication information and/or other information, such as details of physical characteristics, symptoms, or the like. The client device can also include a display for displaying the at least one body status indicator and/or measured body parameter values, although these can alternatively be displayed by the first processing devices, and/or any computers in communication with the first processing devices.

In one example, the processing device(s) generate unique identifiers for each of the plurality of subjects. This can be achieved by the second processing devices, ensuring uniqueness across multiple healthcare practices, or can be achieved by assigning each healthcare practice a unique practice identifier, which is then used locally to create unique identifiers for each patient.

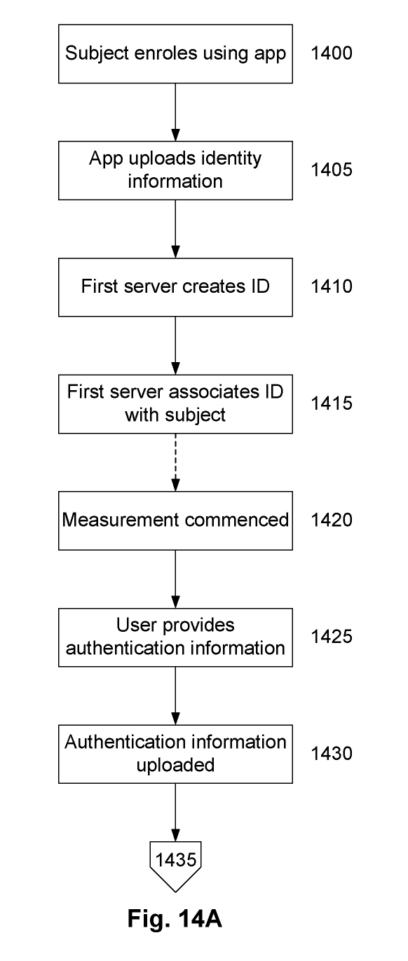

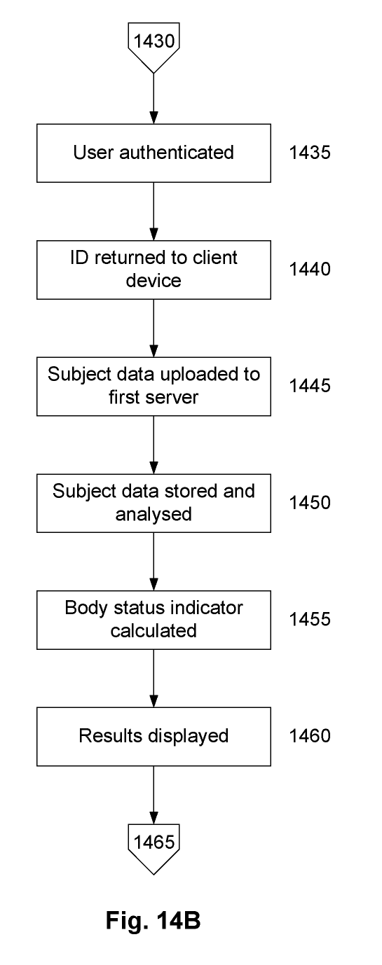

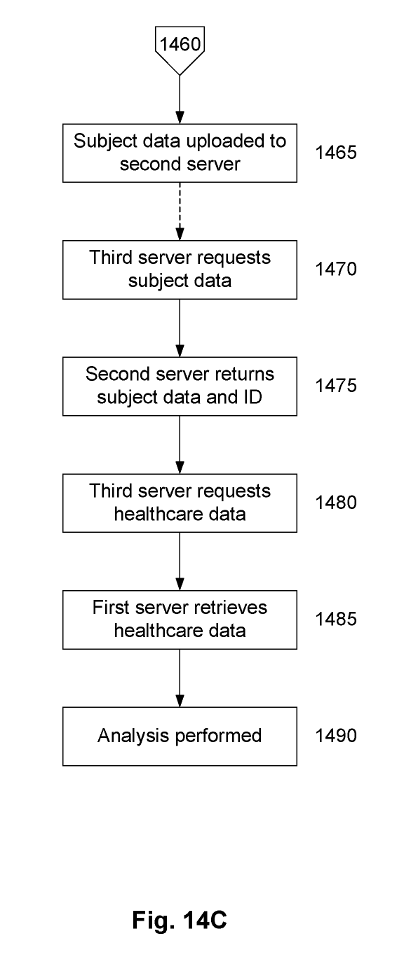

An example of a process for storing and analysing subject data with the architecture of FIG. 12 will now be described in more detail with reference to FIGS. 14A to 14B.

For the purpose of this example, it is assumed that the client device 1230 and first processing system(s) 1250.1 are respectively a tablet 1230 and first server 1250.1 operated by a healthcare provider, at a respective facility where the subject is a patient. This could include a hospital, medical centre, clinician, or the like, although it will be appreciated that similar techniques could be used in other circumstances, and are not necessarily restricted to healthcare scenarios.

The second processing system(s) 1250.2 include second servers 1250.2 operated by a supplier of the measurement devices, and are used to allow subject data from measurement devices in multiple different facilities to be aggregated for collective analysis. Finally, the third processing system(s) 1250.3 are third servers 1250.3 operated by a third party service provided that performs analysis and specifically machine learning based mining of data sets.

In this example, at step 1400 a subject enrols to use the system using an app installed on the client device 1230. This would typically occur when the subject attends the facility, for example as part of a health check-up or the like, and would typically include having the subject provide identity information. This is used to identify the subject, and in particular allows the subject to be linked with existing facility records, if any. The form of the identity information will vary depending on the circumstances, but could include information such as a name, medicare number, social security number or the like. As part of this process, the subject may be required to undergo authentication, and the identity information could include authentication information, such as biometric data or the like.

At step 1405, the identity information is uploaded to the first server 1250.1, which creates a unique identifier (ID) for the subject at step 1410. The unique identifier typically includes a component based on an identifier assigned to the facility by the second server 1250.2, with an additional component to ensure the identifier is unique to the respective subject. The unique ID is then associated with the identity information in the form of an index, which is typically stored in suitable data store, which may form part of, but more typically is separate from the first subject databases 1251.1. The index allows subject data to be stored associated with the ID as opposed to the identity information, meaning the subject can only be identified from the subject data by an entity that has access to the index.

Once the user has been enrolled on the system, measurements can be commenced at step 1420, for example by having the subject interact with the measuring device and/or tablet 1230 in a manner similar to that previously described.

As part of this process, at step 1425, the user provides authentication information, for example by scanning a thumb and/or finger print, undergoing facial recognition, entering a username and password, other like. The authentication information is uploaded to the first server 1250.1, at step 1430, allowing the user to be authenticated at step 1435. In the event that authentication fails, the process is stopped, otherwise at step 1440, the first server 1250.1 determines the identity of the subject and uses this to retrieve the ID from the index, with this being returned to the client device 1230.

At the end of the measurement processes, the client device 1230 uploads the subject data, including any measurement data and/or parameter values derived therefrom, together with the ID at step 1445. The first server 1250.1 stores the subject data and optionally performs analysis, for example to calculate a body status indicator, which can then be displayed at step 1460. As part of this process, historical subject data can be retrieved, for example in order to analyse changes in parameter values. It will be appreciated that the calculation could be performed by the first server 1250.1 and/or by an application installed on the client device 1230, depending on the preferred implementation. Additionally, the results, including the body status indicator could be displayed by the client device 1230, and/or may be accessed from another computing device in communication with the first server 1250.1, for example allowing a clinician to retrieve and view the body status indicator using their own tablet, or computer.

At step 1465, subject data is uploaded to the second server 1250.2 for storage in the one or more second databases 1251.2. This can be completed after each measurement is performed, but more typically is performed periodically, such as at the end of each day and/or in accordance with a defined schedule. This can facilitate the upload of data from multiple facilities, for example to avoid bandwidth restrictions.

At step 1470, the third server 1250.3 requests subject data from the second server 1250.2 in order to perform data mining This can be used to establish reference ranges or signatures, for use in diagnosing body states. In one example, this involves having the third server perform machine learning in order to derive mathematical models that can be used for determining body/disease states. The second server 1250.2 returns requested subject data, together with associated subject identifiers at step 1475.

At step 1480, the third server 1250.3 requests additional healthcare information from the first server 1250.1. In this regard, the second server 1250.2 typically only stores limited information regarding the subject to ensure that the subject is de-identified in the second subject database 1251.2. As information, such as details of medical conditions and/or symptoms may be required by the third server to perform the analysis, this can be requested from the first server 1250.1, which retrieves this, for example from internal records and/or an external medical records system. The healthcare data is returned with the respective ID, allowing this to be matched to the subject data, in turn allowing the analysis to be performed.

Accordingly, the above described system allows measurements to be collected and integrated into an existing healthcare providers records, whilst also allowing de-identified subject data to be made available for analysis.

In the situation in which the measurement device and client device 1230 are used outside of a healthcare provider facility, the system can be implemented in a similar manner, albeit using an index stored at another location. Thus, for example, a first server could be maintained, which is remote from the provider of the measurement devices, but which provides similar functionality to the facility's first server, in terms of assigning IDs and using an index to retrieve an ID based on the subject's identity information. As a further alternative, this functionality could be integrated into the application executed by the client device, for example allowing the client device to store an ID, and forward subject data directly to the second server using the ID. In this instance, each application effectively

It will therefore be appreciated that the above described arrangement provides an improved system for managing subject data, such as electronic medical records.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

Persons skilled in the art will appreciate that numerous variations and modifications will become apparent. All such variations and modifications which become apparent to persons skilled in the art, should be considered to fall within the spirit and scope that the invention broadly appearing before described.

1. A system for managing subject data relating to a body status of a plurality of subjects, the system including:
a) a measuring device that acquires measurement data at least partially indicative of impedance measurements performed on a subject;
b) a client device in communication with the at least one measurement device that:
i) receives measurement data; and
ii) determines identity information indicative of an identity of the subject;
iii) generates collected subject data indicative of at least one of:
(1) the measurement data; and,
(2) measured body parameter values of the subject, the measured body parameters being at least partially derived from impedance measurements performed on the subject; and,
(3) at least one body status indicator at least partially derived from the measured body parameter values; and,
c) one or more processing devices including:
i) one or more first processing devices that:
(1) receive the collected subject data and an indication of the identity information;
(2) retrieve an identifier from an index at least in part using the identity information, the index being indicative of a respective identifier associated with each of a plurality of subjects; and,
(3) store the collected subject data in one or more first subject databases to allow stored subject data to be used in calculating one or more body status indicators indicative of a body status of the subject; and,
ii) one or more second processing devices that:
(1) receive at least some subject data and the respective identifier of at least one subject from the one or more first processing devices; and,
(2) store the subject data in one or more second subject databases in accordance with the respective identifier, to thereby allowing subject data from a plurality of subjects to be analysed.

2. A system according to claim 1, wherein the one or more processing devices:
a) determine a body status indicator to be displayed;
b) retrieve at least some of the stored subject data for the subject in accordance with the determined body status indicator; and,
c) at least one of:
i) generate the body status indicator and provides the body status indicator to the client device; and,
ii) provide retrieved subject data to the client device, the client device being responsive to the retrieved subject data to generate the body status indicator.

3. A system according to claim 1, wherein the identity information includes authentication information supplied by the subject and wherein the one or more first processing devices authenticate the subject to determine the identity of the subject.

4. A system according to claim 3, wherein the client device authenticates the subject using authentication information supplied by the subject and provides the identity information in the form of an indication of the identity of the subject in response to authentication of the subject.

5. A system according to claim 3, wherein the authentication information includes biometric data received via a biometric data reader on the client device.

6. A system according to claim 1, wherein:
a) the client device transfers identity information to the one or more first processing devices;
b) receives the respective identifier from the one or more first processing devices; and,
c) transfers the collected subject data together with the respective identifier to the one or more first processing devices.

7. A system according to claim 1, wherein the client device executes a client device software application that enables encrypted communication with a server application executed by the one or more first processing devices.

8. A system according to claim 1, wherein the one or more processing devices analyse the subject data by performing machine learning using at least subject data for each of a plurality of subjects in order to derive models that can be used in determining body status indicators from measurement data.

9. A system according to claim 1, wherein the system further includes one or more third processing devices, and wherein the third processing devices:
a) retrieve at least some of the stored subject data and respective subject identifiers from the one or more second processing devices for each of a number of subjects;
b) use the respective subject identifiers to retrieve healthcare data for each of a number of subjects from the one or more first processing devices; and,
c) analyse the retrieved subject data and healthcare data to derive one or more models for use in determining body status indicators from measurement data.

10. A system according to claim 1, wherein the one or more first processing devices retrieve healthcare data at least one of:
a) by interfacing with an electronic healthcare record system to retrieve healthcare data relating to a respective subject; and,
b) from the one or more first subject databases.

11. A system according to claim 1, wherein the subject data further includes:
a) an indication of at least one physical characteristic;
b) an indication of at least one symptom; and,
c) the at least one body status indicator.

12. A system according claim 1, wherein the client device presents a user interface to:
a) collect at least one of:
i) authentication information;
ii) an indication of at least one physical characteristic; and,
iii) an indication of at least one symptom; and,
b) display at least one of:
i) the at least one body status indicator; and,
ii) at least one measured body parameter values.

13. A system according to claim 1, wherein the one or more processing devices generate unique identifiers for each of the plurality of subjects.

14. A system according to claim 1, wherein the one or more first processing devices periodically upload subject data to the one or more second processing devices for storage in the one or more second databases.

15. A system according to claim 1, wherein the stored subject data is encrypted within the one or more subject databases and wherein the one or more processing devices includes an encryption module that:
a) encrypts subject data stored in the one or more subject databases; and,
b) decrypts retrieved subject data extracted from the one or more subject databases.

16. A method for managing subject data relating to a body status of a plurality of subjects, the method including:
a) in a measuring device, acquiring measurement data at least partially indicative of impedance measurements performed on a subject;
b) in a client device in communication with the at least one measurement device:
i) receiving measurement data; and
ii) determining identity information indicative of an identity of the subject;
iii) generating collected subject data indicative of at least one of:
(1) the measurement data; and,
(2) measured body parameter values of the subject, the measured body parameters being at least partially derived from impedance measurements performed on the subject; and,
(3) at least one body status indicator at least partially derived from the measured body parameter values; and,
c) in one or more processing devices including:
i) one or more first processing devices, the one or more first processing devices:
(1) receiving the collected subject data and an indication of the identity information;
(2) retrieving an identifier from an index at least in part using the identity information, the index being indicative of a respective identifier associated with each of a plurality of subjects; and,
(3) storing the collected subject data in one or more first subject databases to allow stored subject data to be used in calculating one or more body status indicators indicative of a body status of the subject; and,
ii) one or more second processing devices, the one or more second processing devices:
(1) receiving at least some subject data and the respective identifier for at least one subject from the one or more first processing devices; and,
(2) storing the subject data in one or more second subject databases in accordance with the respective identifier, to thereby allowing subject data from a plurality of subjects to be analysed.

17. (canceled)

18. (canceled)

19. A system for managing subject data relating to a body status of a subject, the system including:
a) one or more subject databases storing subject data for a plurality of subjects, the subject data being indicative of at least one of:
i) body status information; and,
ii) measured body parameter values of the subject; and,
b) one or more processing devices that:
i) receive collected subject data from a client device via a communications network, the client device receiving measurement data indicative of at least one measured body parameter value of a subject from a measuring device, the measuring device being adapted for performing impedance measurements and the measurement data being at least partially indicative of impedance measurements performed on the subject, and the collected subject data being indicative of at least one of:
(1) the measurement data; and,
(2) the at least one measured body parameter value; and,
ii) determine an identity of the subject;
iii) update stored subject data for the subject using the collected subject data and the identity of the subject; and,
iv) retrieve at least some stored subject data for the subject, the retrieved subject data being indicative of previously measured body parameter values for the respective subject and being used to derive at least one body status indicator indicative of a body status of the subject.

20-42. (canceled)

43. A system for monitoring a body status of a subject, the system including a client device having:
c) an input;
d) a display;
e) a memory including a software application; and,
f) a client device processor that:
i) receives measurement data indicative of at least one measured body parameter value of the subject from a measuring device;
ii) determines identity information indicative of an identity of the subject;
iii) provides collected subject data to one or more remote processing devices, the collected subject data being indicative of at least one of:
(1) the measurement data; and,
(2) at least one measured body parameter value derived from the measurement data, the one or more remote processing devices being responsive to the collected subject data to update subject data stored in one or more subject databases, the subject data indicative of:
(a) body status indicator; and,
(b) measured body parameter values of the subject; and,
iv) displays an indication of a body status indicator derived at least in part from measured body parameter values.

44-77. (canceled)

78. A system according to claim 1, wherein:
a) the one or more first processing devices are at least one of:
i) provided at a clinician or other healthcare provider; and,
ii) form part of the clinician or healthcare provider's internal computer systems; and,
b) the one or more second processing devices are provided remotely to the clinician, or other healthcare provider.

79. A system according to claim 1, wherein the second processing is unable to identify the particular subject to which the subject data relates so that the subject data is de-identified in the second processing devices and associated second databases
